(19) United States
(12) Patent Application Publication
(10) Pub. No.: US 2023/0167194 A1
(43) Pub. Date: Jun. 1, 2023

(54) RECOMBINANT BIFUNCTIONAL PROTEIN TARGETING CD47 AND HER2

(71) Applicant: IMMUNEONCO BIOPHARMACEUTICALS (SHANGHAI) INC., Shanghai (CN)

(72) Inventors: Wenzhi TIAN, Shanghai (CN); Song LI, Shanghai (CN)

(21) Appl. No.: 17/820,624

(22) Filed: Aug. 18, 2022

Related U.S. Application Data
(63) Continuation-in-part of application No. 17/407,404, filed on Aug. 20, 2021, now abandoned.
Application No. 17/407,404 is a continuation of application No. 16/535,075, filed on Aug. 8, 2019, now Pat. No. 11,453,724.
(60) Provisional application No. 62/716,356, filed on Aug. 8, 2018.

(51) Int. Cl. C07K 16/32; C07K 14/705; A61P 35/00

(52) U.S. Cl. CPC C07K 16/32; C07K 14/70503; A61P 35/00; A61K 2039/505

(57) ABSTRACT

The present disclosure provides a recombinant fusion protein containing an extracellular Ig-like domain of a signal-regulator protein (SIRP), linked via a linker, to a paratope of an Ig-like anti-HER2 antibody at the N-terminus of a heavy chain or a light chain constituting the paratope. The present disclosure also provides a polynucleotide encoding the recombinant fusion protein, an expression vector containing the polynucleotide, a method for producing the recombinant protein and a method for treating a disease caused by over-expression of CD47 and/or HER2.

### RELATED APPLICATIONS AND INCORPORATION BY REFERENCE

This application is a continuation-in-part application of U.S. patent application Ser. No. 17/407,404 filed Aug. 20, 2021 which is a continuation of U.S. patent application Ser. No. 16/535,075 filed Aug. 8, 2019, which claims priority of U.S. provisional patent application Ser. No. 62/716,356 filed Aug. 8, 2018.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE LISTING

The Sequence Listing XML for this application is labeled 55525-00065SequenceListing which was created on Aug. 4, 2022 and is 30,706 bytes. The entire content of the sequence listing is incorporated herein by reference in its entirety.

### FIELD OF THE INVENTION

The present disclosure relates to a recombinant fusion protein targeting CD47 and HER2, and the preparation and use thereof, especially its use in tumor therapies.

### BACKGROUND OF THE INVENTION

Antibody therapies are approved in various jurisdictions to treat a wide range of cancers, and have significantly improved patient outcomes (Komeev K V et al., (2017) TLR-signaling and proinflammatory cytokines as drivers of tumorigenesis. Cytokine 89: 127-135). Once bound to a cancer antigen, antibodies may induce antibody-dependent cell-mediated cytotoxicity, activate the complement system, or prevent a receptor from interacting with its ligand, all of which may lead to cell death. U.S. FDA-approved antibody drugs include Alemtuzumab, Nivolumab, Rituximab and Durvalumab.

### Tumor Evasion of Immune Surveillance

Cancer cells have developed several mechanisms to evade a host's immune surveillance. For example, many tumor or cancer cells express on their surfaces a high level of CD47, which, by binding to the SIRPα (Signal regulatory protein alpha; also known as SHPS1 and BIT) on the cell surface of macrophages, inhibit phagocytosis of the cancer cells by macrophages.

Cancer cells over-expressing CD47 are found in patients with acute myeloid leukemia (AML), chronic myeloid leukemia (CML), acute lymphoblastic leukemia (ALL), non-hodgkins lymphoma (NHL), multiple myeloma (MM), bladder cancer, ovarian cancer, prostate cancer, lung cancer, colon cancer, breast cancer, and pancreatic cancer. Injection of CD-47 specific antibody that blocks CD47-SIRPα interaction can significantly inhibit tumor growth in tumor-bearing mice, and tumor or cancer cells were eliminated completely when the same antibody was injected into mice carrying human leukemia cells (Theocharides A P A et al., (2012) J.C.Y. J Exp. Med. 209:1883-1899).

### HER2 Associated Tumor and Treatment

HER2, also known as ErbB2, is a member of the human epidermal growth factor receptor family, and encoded by the erythroblastic oncogene B (ERBB2). Overexpression of this oncogene occurs in approximately 15-30% of breast cancers, and is strongly associated with increased disease recurrence and a poor prognosis (Mitri Z et al., (2012) Chemotherapy Research and Practice. Volume 2012, Article ID 743193, 7 pages; Burstein H J, (2005) The New England Journal of Medicine. 353 (16): 1652-4; Tan M, et al., (2007) Advances in Experimental Medicine and Biology. 608: 119-29). Such overexpression is also found in ovarian cancer, stomach cancer, adenocarcinoma of the lung and aggressive forms of uterine cancer.

Monoclonal antibodies have been or are being developed to target HER2. One such antibody, Trastuzumab (Herceptin®), was approved for medical use in the United States in 1998 and has been successfully used in clinical treatment of HER2 positive breast cancers.

### Therapeutic Bi-Specific or Multi-Specific Fusion Proteins/Antibodies

Antibodies have significantly advanced our ability to treat cancers, yet clinical studies have shown many patients do not adequately respond to monospecific therapy. For example, in breast cancer treatment, a substantial percentage of HER2-positive patients do not respond to Trastuzumab treatment due to a number of mechanisms, including the 158F polymorphisms of the FcgRIIIA gene. Additionally, acquired antibody resistance frequently occurs following several cycles of treatment.

Therefore, bispecific or multi-specific antibodies are developed against two or more separate and unique antigens, or different epitopes of the same antigen. For example, some bispecific antibodies are engineered to simultaneously bind a cytotoxic cell and a tumor cell. Such antibodies are capable of blocking multiple tumor cell growth and survival pathways, and/or activating tumor cell killing pathways, and thus have a potential to better inhibit cancer growth.

However, bispecific or multi-specific antibodies present significant design challenges as a number of issues have to be considered, including compatibility of the molecules, the resulting antibody's affinity, stability and pharmaceutical properties. It is well recognized that simply linking antibodies or proteins together does not necessarily result in synergetic/advantageous effects, such modification significantly alters antibody structures and may compromise one another's affinity and/or efficacy (Wang S et al., 2021). In order to optimize in vivo efficacy and pharmaceutical properties, elaborate design and engineering should be given to choice of main and appended binding moieties (sequences), balanced affinities for targets, sites of attachment (N- or C-termini, heavy or light chains), structural stability, linker lengths and sequences (Shim H. 2020).

A recombinant antibody disclosed in the present disclosure, comprising SIRPαD1, linked by a linker, to Erbitux (Cetuximab), has been proved to have inferior anti-tumor activity compared to Erbitux or SIRPαD1-Fc alone in the HT-29 or NCl-H1975 tumor model (see Example 8).

Through diligent efforts, the present inventors, however, have successfully designed a recombinant bispecific protein that accurately targets both CD47 and HER2 and shows better anti-tumor activity than ordinary single antigen targeting antibodies.

### SUMMARY OF THE INVENTION

The present disclosure discloses a recombinant fusion protein, comprising an extracellular Ig-like domain of a signal-regulator protein (SIRP), linked via a linker, to a paratope of an Ig-like anti-HER2 antibody at the N-terminus of a heavy chain or a light chain constituting the paratope, wherein the recombinant fusion protein bind to CD47, HER2 and FcR simultaneously. Binding to CD47s on cancer cells blocks the interaction of CD47s with SIRPs on macrophages and thus releases the check on macrophages by SIRP-mediated inhibitory signals; while binding to HER2s on cancer cells inhibits the uncontrolled tumor cell growth; and at the same time, binding to FcRs on NK cells or macrophages stimulates targeted cancer cell killings by NK cells or macrophages.

In an embodiment, either paratope of the Ig-like anti-HER2 antibody is linked to an extracellular Ig-like domain of signal-regulator protein (SIRP) at the N-terminus of the heavy chain or the light chain constituting the paratope. In an embodiment, each paratope of the Ig-like anti-HER2 antibody is linked to an extracellular Ig-like domain of signal-regulator protein (SIRP) at the N-terminus of the heavy chain or the light chain constituting that paratope. In one embodiment, each paratope of the Ig-like anti-HER2 antibody is linked to an extracellular Ig-like domain of signal-regulator protein (SIRP) at the N-terminus of the heavy chain constituting that paratope. In one embodiment, each paratope of the Ig-like anti-HER2 antibody is linked to an extracellular Ig-like domain of signal-regulator protein (SIRP) at the N-terminus of the light chain constituting that paratope. In one embodiment, one paratope of the Ig-like anti-HER2 antibody is linked to an extracellular Ig-like domain of signal-regulator protein (SIRP) at the N-terminus of the heavy chain constituting that paratope, and the other paratope is linked to an extracellular Ig-like domain of signal-regulator protein (SIRP) at the N-terminus of the light chain constituting that paratope. In some embodiment, one paratope of the anti-HER2 antibodies may be linked to two copies of the extracellular Ig-like domain of signal-regulator protein (SIRP) at the N-terminus of the heavy chain and light chain constituting that paratope.

In one embodiment, the signal-regulatory protein in the recombinant fusion protein may be SIRPα, and the extracellular Ig-like domain of the signal-regulatory protein may be the first extracellular Ig-like domain of SIRPα (SIRPαD1). The extracellular Ig-like domain of the signal-regulatory protein, such as SIRPαD1, can bind to CD47 on the cell surfaces of cancer/tumor cells and thus block the interaction of CD47 with SIRPs on the cell surfaces of macrophages.

In one embodiment, the SIRPαD1 has the nucleic acid sequence and amino acid sequence set forth in SEQ ID NOs: 1 and 2, respectively. In some embodiments, the SIRPαD1 may comprise an amino acid sequence having at least 80%, 85%, 90%, 95%, 98% or 99% identity to SEQ ID NO: 2, wherein the SIRPαD1 can bind to CD47 on the cell surfaces of cancer/tumor cells and block the interaction of CD47 with SIRPs on the cell surfaces of macrophages.

The linker in the recombinant fusion protein may be a peptide of about 5 to 30 amino acid residues. In an embodiment, the linker is a peptide of 10 to 30 amino acid residues. In another embodiment, the linker is a peptide of 15 to 30 amino acid residues. In some embodiments, the linker is -(Gly-Gly-Gly-Gly-Ser)₃- (SEQ ID NO: 4), which may be encoded by SEQ ID NO: 3.

The anti-HER2 antibody may be an isolated monoclonal antibody, such as Trastuzumab, Margetuximab, and antibodies having at least 80%, 85%, 90%, 95%, 98% or 99% amino acid identity to Trastuzumab or Margetuximab while remaining the binding affinity.

The anti-HER2 antibody may be an isolated monoclonal antibody, comprising two heavy chains each having an amino acid sequence of SEQ ID NO: 6, and two light chains each having an amino acid sequence of SEQ ID NO: 8, which may be encoded by nucleic acid sequences of SEQ ID NOs: 5 and 7, respectively. The antigen-binding (Fab) or paratope portion of the anti-HER2 antibody can bind to HER2 on the cell surfaces of cancer/tumor cells and thus prevent uncontrolled tumor/cancer cell growth from occurring, while the Fc portion of the anti-HER2 antibody can bind to FcRs on the cell surfaces of NK cells or macrophages to stimulate cancer cell killings by the NK cells or macrophages. In some embodiments, the heavy chain may comprise an amino acid sequence having at least 80%, 85%, 90%, 95%, 98% or 99% identity to SEQ ID NO: 6, wherein the anti-HER2 antibody is able to bind to HER2 and prevent uncontrolled growth of cancer/tumor cells, and is also able to bind to FcRs on the cell surfaces of NK cells or macrophages and thus activate the NK cells or macrophages for killing the cancer cells. In some embodiments, the light chain may comprise an amino acid sequence having at least 80%, 85%, 90%, 95%, 98% or 99% identity to SEQ ID NO: 8, wherein the anti-HER2 antibody is able to bind to HER2 and prevent occurring of uncontrolled growth of cancer/tumor cells.

The SIRPαD1-Linker-anti-HER2 heavy chain fusion protein may comprise an amino acid sequence of SEQ ID NO: 10, which may be encoded by nucleotide of SEQ ID NO: 9. In some embodiments, the SIRPαD1-Linker-anti-HER2 heavy chain comprises an amino acid sequence having at least 80%, 85%, 90%, 95%, 98% or 99% identity to SEQ ID NO: 10, wherein the SIRPαD1-Linker-anti-HER2 heavy chain together with the light chain of an anti-HER2 antibody can bind to CD47, HER2 and FcR, i) blocking the interaction of CD47 on cancer cells with SIRPs on macrophages; ii) inhibiting uncontrolled cancer/tumor cell growth; and iii) stimulating cancer cell killings by NK cells or macrophages.

In one embodiment, the SIRPαD1-Linker-anti-HER2 light chain fusion protein comprises an amino acid sequence of SEQ ID NO: 12, which may be encoded by nucleotide of SEQ ID NO: 11. In some embodiments, the SIRPαD1-Linker-anti-HER2 light chain comprises an amino acid sequence having at least 80%, 85%, 90%, 95%, 98% or 99% identity to SEQ ID NO: 10, wherein the SIRPαD1-Linker-anti-HER2 light chain together with the heavy chain of an anti-HER2 antibody can bind to CD47, HER2 and FcR, i) blocking the interaction of CD47 on cancer cells with SIRPs on macrophages; ii) inhibiting uncontrolled cancer/tumor cell growth; and iii) stimulating cancer cell killings by NK cells or macrophages.

A nucleic acid molecule encoding the recombinant fusion protein of the present disclosure is also provided, as well as an expression vector comprising the nucleic acid and a host cell comprising the expression vector.

A method for preparing the recombinant fusion protein using the host cell comprising the expression vector is also provided, and comprises steps of (i) expressing the recombinant fusion protein in the host cell and (ii) isolating the recombinant fusion protein from the host cell.

In another respect, the present disclosure provides a pharmaceutical composition, comprising the recombinant fusion protein of the present disclosure, and at least one pharmaceutically acceptable carrier. The pharmaceutical composition may further comprise at least one adjuvant.

In another aspect, the present disclosure provides a method for treating a disease caused by over-expression of CD47 and/or HER2, comprising administering to a patient or a subject in need thereof a therapeutically effective amount of the pharmaceutical composition of the present disclosure.

In one embodiment, the present disclosure provides the use of the recombinant fusion protein in the manufacture of a pharmaceutical composition for the treatment of a disease caused by over-expression of CD47 and/or HER2.

In one embodiment, the method of the present disclosure is for treating a disease selected from the group consisting of acute myelocytic leukemia (AML), chronic myelocytic leukemia (CML), acute lymphoblastic leukemia (ALL), non-Hodgkin's lymphoma (NHL), multiple myeloma (MM), bladder cancer, ovarian cancer, gastric cancer, prostate cancer, lung cancer, colon cancer, breast cancer, pancreatic cancer, and renal cell carcinoma. In one embodiment, the present disclosure provides a method for treating Crohn's disease, allergic asthma or rheumatoid arthritis.

Other features and advantages of the instant disclosure will be apparent from the following detailed description and examples, which should not be construed as limiting. The contents of all references, Genbank entries, patents and published patent applications cited throughout this application are expressly incorporated herein by reference.

### BRIEF DESCRIPTION OF THE DRAWINGS

FIG. 1 is a schematic diagram of the structures of the recombinant fusion proteins of the present disclosure.

FIG. 2 shows the binding activities of IMM2901 and IMM2902 to CD47.

FIG. 3 shows the binding activities of IMM2901 and IMM2902 to HER2.

FIG. 4 shows IMM2902 induced phagocytosis of HL-60 leukemia cells.

FIG. 5 shows IMM2902's ADCC activity.

FIG. 6 shows IMM2902 induced HER2 internalization.

FIG. 7 shows in vivo therapeutic efficacy of IMM2902 in BT-474 breast cancer xenograft model.

FIG. 8 shows in vivo therapeutic efficacy of IMM0404 in HT-29 colon cancer xenograft model.

FIG. 9 shows in vivo therapeutic efficacy of IMM0404 in NCI-H1975 non-small cell lung cancer xenograft model.

FIG. 10 shows the binding activity of IMM2902 to CD47⁺HER2⁺NCl-N87 gastric cancer cells.

FIG. 11 shows the binding activity of IMM2902 to CD47+HER2+SK-OV-3 ovarian cancer cells.

FIG. 12 shows the ability of IMM2902 to induce FcR-TANK cell-mediated ADCC against CD47⁺HER2⁺SK-OV-3 ovarian cancer cells.

FIG. 13 shows the ability of IMM2902 to induce FcR-TANK cell-mediated ADCC against CD47⁺HER2⁺NCl-N87 gastric cancer cells.

FIG. 14 shows the ability of IMM2902 to induce FcR_158F-TANK cell-mediated ADCC against NCl-N87 gastric cancer cells.

FIG. 15 shows the ability of IMM2902 to induce FcR_158F-TANK cell-mediated ADCC against NCl-N87 gastric cancer cells.

FIG. 16 shows the ability of IMM2902 to induce phagocytosis of SK-OV-3 ovarian cancer cells by THP-1 cells.

FIG. 17 shows the tumor sizes of gastric tumor-bearing mice treated with IMM2902.

FIG. 18 shows the tumor sizes of gastric tumor-bearing mice treated with IMM2902.

### DETAILED DESCRIPTION OF THE INVENTION

There are principally three different approaches to targeting two or more pharmacologies of tumor growth. Most commonly, patients can be given a cocktail of two or more different drugs. Although this option allows for maximal flexibility with respect to possible drug combinations and different dosages, it suffers from (a) potentially poor adherence to treatment by the patient because of the increased pill burden and the different dosing schedules for the individual drugs, (b) possible incompatibilities because of drug-drug interactions, and (c) increased risk of drug side effects. These problems may reduce the effectiveness of therapy and hamper the attainment of treatment goals particularly in the management of chronic diseases such as cancer.

The second approach relies on the use of fixed-dose combinations of drugs in a single dosage form. This approach reduces pill burden, resulting in improved patient compliance. The disadvantage of fixed-dose combinations is primarily the limited choice of possible dose ratios between the active ingredients, which makes it more difficult to properly titrate the individual patient to maximum efficacy with minimal adverse effects. In addition, different pharmacokinetic properties of the components in the combination might lead to a complex temporal mismatch in pharmacodynamic effects at the individual targets thereby compromising overall efficacy.

The third approach is the use of multifunctional drugs that combine two or more pharmacologies in a single entity. The design and validation of such multifunctional entities are more complex and require substantial investigation into the optimal ratio of target activities. Multifunctional molecules may also be amenable to fixed dose combination with other drugs thereby combining three or even four pharmacologies in a single pill to produce further increments in efficacy.

A recombinant bispecific or multi-specific protein against two or more targets is a multifunctional drug, which does not necessarily show superior anti-tumor activity compared to ordinary single antigen targeting antibodies. For example, as shown in the Example 8 below, a recombinant antibody comprising SIRPαD1, linked by a linker, to Erbitux (Cetuximab), an anti-EGFR antibody, had lower anti-tumor activity than Erbitux or SIRPαD1-Fc in the HT-29 or NCl-H1975 tumor model. Smart designs are needed for a recombinant protein to provide synergistic effects.

Through diligent experimentation, the present inventors have invented a novel recombinant fusion protein, which can attack tumors, via three mechanisms of actions, one to release the check on macrophages by SIRP-mediated inhibitory signals, one to control HER2 signaling mediated tumor/cancer cell proliferation, the third to stimulate cancer cell killings by NK cells and/or macrophages.

The recombinant fusion protein of the present disclosure comprises comprising an extracellular Ig-like domain of a signal-regulator protein (SIRP), linked via a linker, to a paratope of an Ig-like anti-HER2 antibody at the N-terminus of a heavy chain or a light chain constituting the paratope, wherein the recombinant fusion protein bind to CD47, HER2 and FcR simultaneously, i) binding to CD47s on cancer cells to block the interaction of CD47s with SIRPs on macrophages and thus releasing the check on macrophages by SIRP-mediated inhibitory signals; ii) binding to HER2 on cancer cells and thus inhibiting uncontrolled tumor/cancer cell growth; and iii) binding to FcRs on NK cells or macrophages to stimulate cancer cell killings by NK cells or macrophages. In an embodiment, either paratope of the Ig-like anti-HER2 antibody is linked to an extracellular Ig-like domain of signal-regulator protein (SIRP) at the N-terminus of the heavy chain or the light chain constituting the paratope. In an embodiment, each paratope of the Ig-like anti-HER2 antibody is linked to an extracellular Ig-like domain of signal-regulator protein (SIRP) at the N-terminus of the heavy chain or the light chain constituting that paratope. In one embodiment, each paratope of the Ig-like anti-HER2 antibody is linked to an extracellular Ig-like domain of signal-regulator protein (SIRP) at the N-terminus of the heavy chain constituting that paratope. In one embodiment, each paratope of the Ig-like anti-HER2 antibody is linked to an extracellular Ig-like domain of signal-regulator protein (SIRP) at the N-terminus of the light chain constituting that paratope. In one embodiment, one paratope of the Ig-like anti-HER2 antibody is linked to an extracellular Ig-like domain of signal-regulator protein (SIRP) at the N-terminus of the heavy chain constituting that paratope, and the other paratope is linked to an extracellular Ig-like domain of signal-regulator protein (SIRP) at the N-terminus of the light chain constituting that paratope. In some embodiment, one paratope of the anti-HER2 antibodies may be linked to two copies of the extracellular Ig-like domain of signal-regulator protein (SIRP) at the N-terminus of the heavy chain and light chain constituting that paratope.

The three main components contained in the fusion protein of the present disclosure are an extracellular Ig-like domain of a signal-regulator protein (SIRP), a linker, and an anti-HER2 antibody. A person of ordinary skills in the art will recognize that there are many design choices for selecting the above three components. Preferably, human-derived sequence is used in human cancer therapies, as the strong immunogenicity of the proteins or peptides from non-human animals may lead to allergy and other adverse effects. However, other animal proteins or peptides, humanized if appropriate, may also be used in the present disclosure based on different application purposes.

Any extracellular Ig-like domain of any SIPR (SIRPα, SIRPβ, and SIRPγ) capable of binding with CD47 may be selected for construction of the fusion protein. In one embodiment, the signal-regulatory protein in the recombinant fusion protein is SIRPα, and the extracellular Ig-like domain of the signal-regulatory protein is the first extracellular Ig-like domain of SIRPα (SIRPαD1).

In one embodiment, the recombinant fusion protein comprises SIRPαD1 having the nucleic acid sequence and amino acid sequence set forth in SEQ ID NOs: 1 and 2, respectively. In another embodiment, the SIRPαD1 may comprise an amino acid sequence having at least 80%, 85%, 90%, 95%, 98% or 99% identity to SEQ ID NO: 2, wherein the SIRPαD1 can bind to CD47 on the cell surface of cancer/tumor cells and block the interaction of CD47 with SIRPs on the cell surfaces of macrophages.

Linkers serve primarily as a spacer between the extracellular Ig-like domain of SIRP and the N-terminus of the heavy or light chain of an anti-HER2 antibody. The linker may be made up of amino acids linked together by peptide bonds, preferably from 5 to 30 amino acids linked by peptide bonds, wherein the amino acids are selected from the 20 naturally occurring amino acids. When two copies of the SIRP extracellular Ig-like domain are linked to one paratope of the anti-HER2 antibody at the N-terminus of the heavy chain and the light chain constituting that paratope, a relatively long linker, maybe of 10 or more, or even 15 or more amino acid resides in length, may be needed to avoid possible stereo hindrance. One or more of these amino acids may be glycosylated, as is understood by those of skill in the art. In one embodiment, the 5 to 30 amino acids may be selected from glycine, alanine, proline, asparagine, glutamine, serine and lysine. In one embodiment, a linker is made up of a majority of amino acids that are sterically unhindered, such as glycine and alanine. Exemplary linkers are polyglycines (particularly (Glys, (Gly)₈, poly(Gly-Ala), and polyalanines. One exemplary suitable linker as shown in the Examples below is poly(Gly-Ser), such as -(Gly-Gly-Gly-Gly-Ser)₃- (SEQ ID NO: 21).

Linkers may also be non-peptide linkers. For example, alkyl linkers such as —NH—, —(CH₂)s-C(O)—, wherein s=2-20 can be used. These alkyl linkers may further be substituted by any non-sterically hindering group such as lower alkyl (e.g., C₁₋₄) lower acyl, halogen (e.g., CI, Br), CN, NH₂, phenyl, etc.

Any anti-HER2 antibody, especially any Ig-like anti-HER2 antibody, may be used in the formation of the fusion protein of the present disclosure. The anti-HER2 antibody may be an isolated monoclonal antibody such as Trastuzumab and Margetuximab.

In some embodiments, the anti-HER2 antibody is an isolated monoclonal antibody comprising two heavy chains each having an amino acid sequence of SEQ ID NO: 6, and two light chains each having an amino acid sequence of SEQ ID NO: 8, which two may be encoded by nucleic acid sequences of SEQ ID NOs: 5 and 7, respectively. The Fab or paratope portion of the anti-HER2 antibody can bind to HER2 on the cell surfaces of cancer/tumor cells and thus prevent the occurring of uncontrolled growth of cancer/tumor cells, while the Fc portion of the anti-HER2 antibody can bind to FcRs on the cell surfaces of NK cells or macrophages to stimulate cancer cell killings by the NK cells or macrophages. In some embodiments, the heavy chain may comprise an amino acid sequence having at least 80%, 85%, 90%, 95%, 98% or 99% identity to SEQ ID NO: 6, wherein the anti-HER2 antibody is able to bind to HER2 and prevent the occurring of uncontrolled growth of cancer/tumor cells, and is also able to bind to FcRs on the cell surfaces of NK cells or macrophages and thus activate the NK cells or macrophages for killing the cancer cells. In some embodiments, the light chain may comprise an amino acid sequence having at least 80%, 85%, 90%, 95%, 98% or 99% identity to SEQ ID NO: 8, wherein the anti-HER2 antibody is able to bind to HER2 and prevent occurring of uncontrolled growth of cancer/tumor cells.

As described above, one or two copies of the SIRP extracellular Ig-like domain especially SIRPαD1 can be linked to either or each paratope of the anti-HER2 at the N-terminal of the heavy chain and/or the light chain constituting the specific paratope.

Also, the present disclosure provides a polynucleotide molecule encoding the recombinant fusion protein and an expression vector expressing the recombinant bi-functional fusion protein. Examples of vectors include but are not limited to plasmids, viral vectors, yeast artificial chromosomes (YACs), bacterial artificial chromosomes (BACs), transformation-competent artificial chromosomes (TACs), mammalian artificial chromosomes (MACs) and human artificial episomal chromosomes (HAECs).

The present disclosure provides host cells comprising the above expression vectors. The host cells may be transformed or transfected with the expression vectors. Suitable host cells include Escherichia coli, yeasts and other eukaryotes. Preferably, Escherichia coli, yeast or mammalian cell lines (such as COS or CHO) are used.

In another aspect, the present disclosure provides a pharmaceutical composition comprising the fusion protein of the present disclosure formulated together with a pharmaceutically acceptable adjuvant. The composition may optionally contain one or more additional pharmaceutically active ingredients, such as another antibody or a drug. The pharmaceutical compositions of the disclosure also can be administered in a combination therapy with, for example, another immune-stimulatory agent, anti-cancer agent, or a vaccine.

The pharmaceutical composition can comprise any number of excipients. Excipients that can be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003), the disclosure of which is incorporated herein by reference.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in injection. For example, the vehicle or carrier may be neutral buffered saline or saline mixed with serum albumin. Other exemplary pharmaceutical compositions comprise Tris buffers, or acetate buffers, which may further include sorbitol or a suitable substitute thereof. In one embodiment of the present disclosure, compositions may be prepared for storage by mixing the selected composition having the desired degree of purity with optional formulation agents (Remington's Pharmaceutical Sciences, supra) in the form of a lyophilized cake or an aqueous solution. Further, the therapeutic composition may be formulated as a lyophilizate using appropriate excipients such as sucrose.

Preferably, the pharmaceutical composition is suitable for intravenous, intramuscular, subcutaneous, parenteral, spinal or epidermal administration (e.g., by injection or infusion). Depending on the route of administration, the active molecule can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, an antibody of the disclosure can be administered via a non-parenteral route, such as a topical, epidermal or mucosal route of administration, e.g., intranasally, orally, vaginally, rectally, sublingually or topically.

Pharmaceutical compositions can be in the form of sterile aqueous solutions or dispersions. They can also be formulated in a microemulsion, liposome, or other ordered structure suitable to high drug concentration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01% to about 99% of active ingredient, preferably from about 0.1% to about 70%, most preferably from about 1% to about 30% of active ingredient in combination with a pharmaceutically acceptable carrier.

Dosage regimens are adjusted to provide the optimum desired response (e.g., a therapeutic response). For example, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. Alternatively, the fusion protein can be administered as a sustained release formulation, in which case less frequent administration is required.

For administration of the fusion protein, the dosage ranges from about 0.0001 to 100 mg/kg, and more usually 0.01 to 10 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration twice per week, once per week, once every two weeks, once every three weeks, once every four weeks, once a month, once every 3 months or once every three to 6 months. Preferred dosage regimens for the fusion protein of the disclosure include 3 mg/kg body weight or 6 mg/kg body weight via intraperitoneal administration, with the antibody being given using one of the following dosing schedules: (i) every four weeks for six dosages, then every three months; (ii) every three weeks; (iii) 3 mg/kg body weight once followed by 1 mg/kg body weight every three weeks; (vi) 6 mg/kg body weight, one dosage per week. In some methods, dosage is adjusted to achieve a plasma antibody concentration of about 1-1000 μg/ml and in some methods about 25-300 μg/ml.

A “therapeutically effective dosage” of a fusion protein of the disclosure preferably results in a decrease in severity of disease symptoms, an increase in frequency and duration of disease symptom-free periods, or a prevention of impairment or disability due to the disease affliction. For example, for the treatment of tumor-bearing subjects, a “therapeutically effective dosage” preferably inhibits tumor growth by at least about 40%, more preferably by at least about 60%, even more preferably by at least about 80%, and still more preferably by at least about 99% relative to untreated subjects. A therapeutically effective amount of a fusion protein of the present disclosure can decrease tumor size, or otherwise ameliorate symptoms in a subject, which is typically a human or can be another mammal.

The pharmaceutical composition can be a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices (e.g., U.S. Pat. Nos. 5,399,163; 5,383,851; 5,312,335; 5,064,413; 4,941,880; 4,790,824; and 4,596,556); (2) micro-infusion pumps (U.S. Pat. No. 4,487,603); (3) transdermal devices (U.S. Pat. No. 4,486,194); (4) infusion apparatuses (U.S. Pat. Nos. 4,447,233 and 4,447,224); and (5) osmotic devices (U.S. Pat. Nos. 4,439,196 and 4,475,196); the disclosures of which are incorporated herein by reference.

In certain embodiments, the fusion protein of the disclosure can be formulated to ensure proper distribution in vivo. For example, to ensure that the therapeutic fusion proteins of the disclosure cross the blood-brain barrier, they can be formulated in liposomes, which may additionally comprise targeting moieties to enhance selective transport to specific cells or organs. See, e.g. U.S. Pat. Nos. 4,522,811; 5,374,548; 5,416,016; and 5,399,331.

A gene therapy in vivo is also envisioned wherein a nucleic acid molecule encoding the recombinant fusion protein of the present disclosure, or a derivative thereof is introduced directly into the subject. For example, a nucleic acid sequence encoding a recombinant fusion protein of the present disclosure is introduced into target cells via local injection of a nucleic acid construct with or without an appropriate delivery vector, such as an adeno-associated virus vector. Alternative viral vectors include, but are not limited to, retroviruses, adenovirus, herpes simplex vims and papilloma virus vectors. Physical transfer of the virus vector may be achieved in vivo by local injection of the desired nucleic acid construct or other appropriate delivery vector containing the desired nucleic acid sequence, liposome-mediated transfer, direct injection (naked DNA), or microparticle bombardment (gene-gun).

The compositions of the present disclosure may be used alone or in combination with other therapeutic agents to enhance their therapeutic effects or decrease potential side effects.

Another object of the present disclosure is to provide a method for preparing the above recombinant fusion protein and the pharmaceutical composition comprising the same. In one embodiment, the method comprises (1) providing an protein-encoding polynucleotide molecule; (2) constructing an expression vector comprising the polynucleotide molecule of (1); (3) transfecting or transforming suitable host cells with the expression vector of (2) and cultivating the host cells to express the protein; and (4) purifying the protein. The preparation may be carried out with well-known technologies by an ordinarily skilled artisan.

Another object of the present disclosure is to provide a method of treating cancer using the pharmaceutical composition of the present disclosure, comprising administrating an effective amount of the aforementioned pharmaceutical composition to the patients or subjects in need thereof. In one embodiment, the pharmaceutical composition is used to treat CD47 and/or HER2-overexpressing tumors or cancers, including but not limited to acute myeloid leukemia (AML), chronic myeloid leukemia (CML), acute lymphoblastic leukemia (ALL), non-hodgkins lymphoma (NHL), multiple myeloma (MM), bladder cancer, ovarian cancer, gastric cancer, prostate cancer, lung cancer, colon cancer, breast cancer, pancreatic cancer and renal cancer.

In one embodiment, the diseases related to over-expressions of CD47 and/or HER2 include but are not limited to Crohn's disease, allergic asthma, and rheumatoid arthritis.

The present disclosure is now further described with the non-limiting examples below.

### EXAMPLES

In the examples below, IMM29 refers to a HER2-specific antibody. This antibody has two heavy chains each having an amino acid sequence of SEQ ID NO: 6, and two light chains each having an amino acid sequence of SEQ ID NO: 8, which two may be encoded by nucleic acid sequences of SEQ ID NOs: 5 and 7, respectively.

IMM2901 is a recombinant fusion protein capable of binding to CD47 and HER2, containing two SIRPαD1s each linked via a GS-linker, to IMM29 at the N-terminus of each heavy chain, wherein the SIRPαD1 has an nucleic acid sequence and amino acid sequence of SEQ ID NO: 1 and SEQ ID NO: 2, respectively, and the linker having an amino acid sequence of SEQ ID NO: 4, which can be encoded by the nucleic acid sequence of SEQ ID NO: 3.

IMM2902 is also a recombinant fusion protein capable of binding to CD47 and HER2, and differs from IMM2901 in that each SIRPαD1 is linked via a GS-linker, to IMM29 at the N-terminus of each light chain.

IMM01 is a fusion protein capable of binding to CD47, consisting of SIRPαD1 linked to an Fc fragment, which was described in WO2016169261. The nucleic acid sequence and amino acid sequence of this fusion protein are set forth in SEQ ID NO: 13 and SEQ ID NO: 14, respectively.

IMM0404 is a recombinant fusion protein, containing two SIRPαD1s each linked via a GS-linker, to an anti-EGFR antibody at the N-terminus of each heavy chain. The SIRPαD1-GS-linker-anti-EGFR heavy chain has a nucleic acid sequence and amino acid sequence of SEQ ID NO: 15 and SEQ ID NO: 16, respectively. The light chain of the anti-EGFR antibody has an amino acid sequence of SEQ ID NO: 18, which may be encoded by nucleic acid sequences of SEQ ID NO: 17.

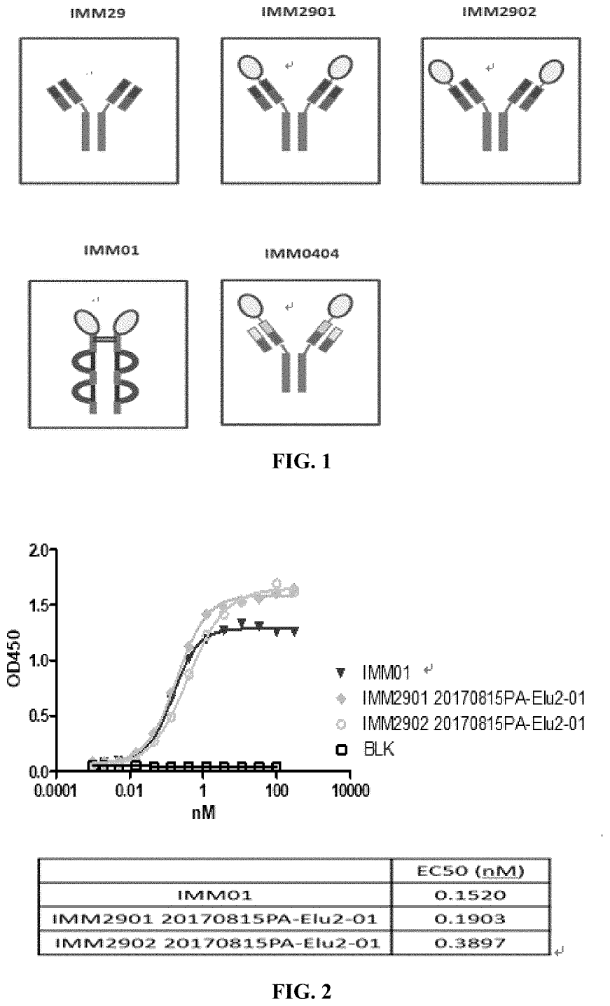

The structures of these proteins can be found in FIG. 1.

### Example 1. Construction of Vectors Expressing IMM29, IMM2901, IMM2902, IMM01 and IMM0404

### 1.1 IMM29

The full-length coding sequence of IMM29 was designed artificially. Specifically, the coding sequences of both the heavy chain and the light chain variable regions were derived from Herceptin (Trastuzumab). 57 nucleotides encoding the signal peptide of mouse IgG1 heavy chain (SEQ ID NO.: 19) were added to the 5′ end of the heavy chain-coding sequence (SEQ ID NO.: 5) or the light chain-coding sequence (SEQ ID NO.:7), and a Kozak sequence (SEQ ID NO.: 20: GCCGCCACC) was added to the 5′ end of the signal peptide sequence. Then, HindIII and NheI restriction sites were added to the 5′ and 3′ ends of the resulting heavy chain sequence, and HindIII and the XbaI restriction sites were added to the 5′ and 3′ ends of the resulting light chain sequence. The two resulting sequences were synthesized by Genscript (ID #: T84300 (heavy chain); T85555 (light chain)) and subcloned, respectively, into the pMac-H and pMac-L vectors.

### 1.2 IMM2901

The expression vector for the light chain of IMM2901 is identical to that of IMM29. For the heavy chain vector construction, the coding sequence of the first extracellular domain of SIRPα (SIRPαD1) (SEQ ID NO.:1) was linked through a GS-linker (SEQ ID NO.:3) to the N terminal of the heavy chain coding sequence of IMM29 (SEQ ID NO.:5) (totally SEQ ID NO.: 9). 57 nucleotides encoding the signal peptide of mouse IgG1 heavy chain (SEQ ID NO.:19) were added to the 5′ end of SIRPαD1-coding sequence, and a Kozak sequence (SEQ ID NO.: 20: GCCGCCACC) was added to the 5′ end of the signal peptide sequence. Lastly, HindIII and NheI restriction sites were added to the 5′ and 3′ ends of the resulting sequence, respectively. The resulting sequence was synthesized by Convenience Biology (ID #: Y0000506-1-A10863) and subcloned into the pMac-H vector.

### 1.3 IMM2902

The expression vector for the heavy chain of IMM2902 is identical to that of IMM29. For the light chain vector construction, the coding sequence of the first extracellular domain of SIRPα (SIRPαD1) (SEQ ID NO.:1) was linked through a GS-linker (SEQ ID NO.:3) to the N terminal of the light chain coding sequence of IMM29 (SEQ ID NO.:7) (totally SEQ ID NO.: 11). 57 nucleotides encoding the signal peptide of mouse IgG1 heavy chain (SEQ ID NO.:19) were added to the 5′ end of SIRPαD1-coding sequence, and a Kozak sequence (SEQ ID NO.: 20: GCCGCCACC) was added to the 5′ end of the signal peptide sequence. Lastly, HindIII and XbaI restriction sites were added to the 5′ and 3′ ends of the resulting sequence, respectively. The resulting sequence was synthesized by Convenience Biology (ID #: Y0000506-2-A10868) and subcloned into the pMac-L vector.

### 1.4 IMM01

The expression cassette of SIRPαD1-Fc was designed by sequentially connecting a Kozak sequence (SEQ ID NO.: 20: GCCGCCACC) with the coding sequence of the signal peptide (SEQ ID NO.:19) and SIRPαD1-Fc (SEQ ID NO.:13). HindIII and EcoRI restriction sites were respectively added to the 5′ and 3′ ends of the resulting sequence, which was synthesized by Convenience Biology (ID #: CN1418-F9043) and subcloned into the pMac-Fc vector. 1.5 IMM0404

The expression cassette for the light chain of IMM0404 was designed by sequentially connecting a Kozak sequence (SEQ ID NO.: 20: GCCGCCACC) with the coding sequence of the signal peptide (SEQ ID NO.:19) and the light chain of an anti-EGFR antibody (Erbitux (Cetuximab)) (SEQ ID NO.:17). HindIII and XbaI restriction sites were respectively added to the 5′ and 3′ ends of the resulting sequence, which was synthesized by Convenience Biology (ID #: NJ0719028J_A6315) and subcloned into the pMac-L vector. For the heavy chain vector construction, the coding sequence of the first extracellular domain of SIRPα (SIRPαD1) was linked through a GS-linker to the N terminal of the heavy chain coding sequence of an EGFR-specific antibody, the SIRPαD1-GS-linker-heavy chain was encoded by SEQ ID NO.:15. 57 nucleotides encoding the signal peptide of mouse IgG1 heavy chain (SEQ ID NO.:19) were added to the 5′ end of SIRPαD1-GS-linker-heavy chain (SEQ ID NO.: 15), and a Kozak sequence (SEQ ID NO.: 20: GCCGCCACC) was added to the 5′ end of the signal peptide sequence. Lastly, HindIII and NheI restriction sites were added to the 5′ and 3′ ends of the resulting sequence, which was synthesized by Genscript (ID #: M17025) and subcloned into the pMac-H vector.

### Example 2. Protein Expression and Purification

To manufacture the desired proteins, the expression vectors of Example 1 were electroporated into Chinese Hamster Ovary (CHO) cells (ATCC, Cat #CCL-61) which were subjected to several rounds of pressure selection of neomycin. The selected stable cells were adapted to a serum-free Balan CD CHO Growth A medium (Irvine Scientific, Cat #941 20). For protein expression, cells were seeded in a 3 L bioreactor and cultured in a fed-batch process. When the cell viability dropped to ˜80%, reaction in the bioreactor was terminated, the cell culture supernatant was harvested and subjected to protein purification by affinity chromatography. The purity of recombinant protein was above 95%, and the content of endotoxin was below 0.5 U/g.

### Example 3. IMM2901 and IMM2902 Bound to CD47 or HER2

CD47 or HER2 binding capacities of the recombinant proteins were measured by the enzyme-linked immunosorbent assay (ELISA). Recombinant Human CD47 (Lot #LC10DE2004, Sino Biologicals) and ErbB2 (Lot #LC11MC0201, Sino Biologicals) were, respectively, prepared in coating buffer (Product code: 1001329288 C3041-100CAP, Sigma-Aldrich Co.) and transferred to the ELISA plates (Cat #442404, Nunc™) at 50 ng/well. The plates were placed in 4° C. refrigerator overnight. When assays were performed, plates were washed for three times with PBS containing 0.05% of Tween-20 (PBS-T) before the titrated proteins were added, and the plates were incubated at room temperature for 1 hour. The plates were washed again for 5 times with PBS-T, and then HRP-Rabbit Anti-Human IgG Fc (Cat #:309-036-008, Jackson ImmunoResearch Lab) was added to the plates and incubated at room temperature for one hour. After the plates were washed for 5 times with PBS-T, and substrates were added to the plates which were read in a plate reader after the color changing was stopped by 1N H₂SO₄.

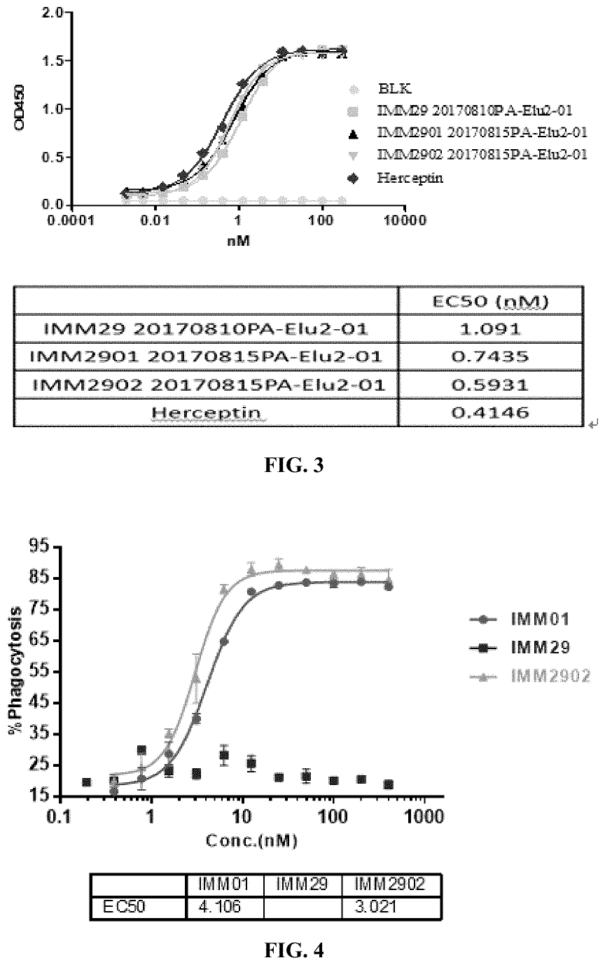

IMM2901 and IMM2902 bound to CD47 with an EC₅₀ value of 0.1903 nM and 0.3894, respectively (FIG. 2), and bound to HER2 with an EC₅₀ value of 0.7435 nM and 0.5931 nM, respectively (FIG. 3), a bit interior to the traditional single antigen targeting antibodies.

### Example 4. IMM2902 Activated Phagocytosis of HL-60

Mouse macrophage cell line Ana-1 was seeded in a 96-well cell culture plate, 1×10⁵ cells per well, and cultured for 16-18 hours at 37° C. and 5% CO₂. Target cells (HL-60) were labeled with CF SE, and then incubated with serially diluted IMM2902, or control proteins for 45 minutes. The target cell solutions containing the test proteins were transferred to the plate containing Ana-1 cells, the ratio of the number of Ana-1 cells to HL-60 cells was 1:3. The mixture was cultured for 2 hours at a cell culture incubator and then subject to FACS analysis for density of CFSE in Ana-1 cells.

As shown in FIG. 4, IMM2902 activated a high level of phagocytosis of tumor cells.

### Example 5. IMM2902 had High Antibody-Dependent Cell-Mediated Cytotoxicity (ADCC)

CFSE-labeled BT-474 cells (used as target cells) were mixed with NK92MI cells (effector cells) stably expressing FcγRIIIa at a 1:2 ratio, and the mixed cells were cultured for 4 hours at 37° C. under 5% CO₂ in the presence of serially diluted IMM2902 or control proteins. Then 5 g/ml propidium iodide (PI) (Sigma, Cat#P4170) was added to the cell culture at a concentration of 5 μg/ml, and the cell culture was subjected to FACS analysis for PI signals. Percentage of cell lysis mediated by ADCC was calculated based on the following formula:

% Lysis=(% PI Positive Cell with IMM2902 or control proteins−% PI Positive Cell with negative control protein)/(100−% PI Positive Cell with negative control protein)*100

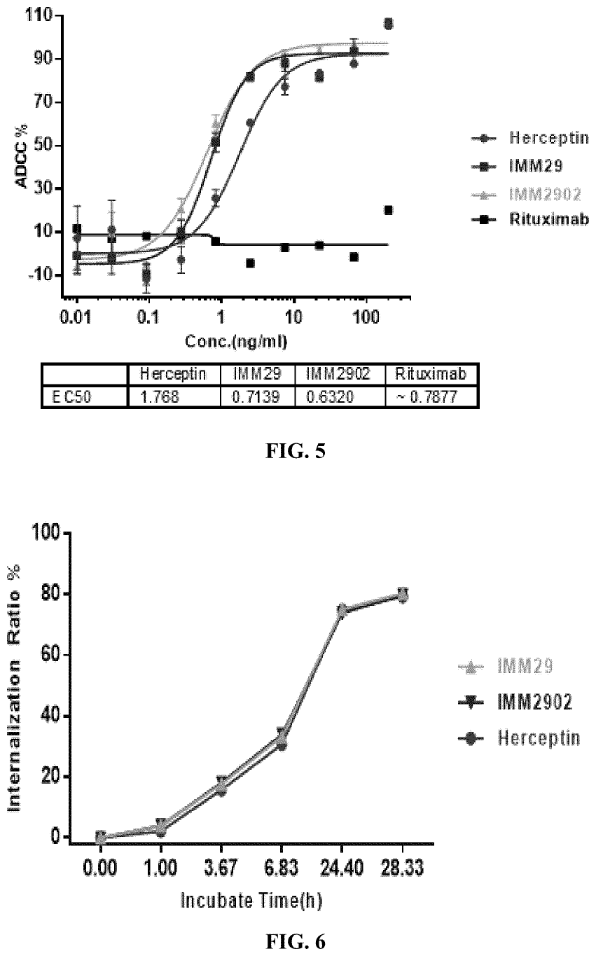

As shown in FIG. 5, IMM2902 had a comparable or better ADCC activity compared to IMM29 and Herceptin.

### Example 6. IMM2902 Induced HER2 Internalization

1×106 of BT-474 cells in 200 L of DMEM medium containing 5% of FBS were seeded in a 96-well cell culture plate and incubated at 37° C. and 5% CO₂ overnight. On the next day, the plate was taken out of the incubator and the medium was replaced with fresh medium containing titrated proteins. The plate was incubated for further 4 hours in the cell culture incubator before the cells were washed and stained with FITC-conjugated antibody specific for the Fc portion of human IgG. Percentage of HER2 receptor internalized was calculated based on the formula below:

Internalization Ratio=(1−MFI/MFIt=0)*100%

MFI: mean fluorescence intensity

According to FIG. 6, IMM2902 was able to induce HER2 internalization in an equivalent level to IMM29 or Herceptin.

### Example 7. IMM2902 had Good Anti-Tumor Effect

BT-474 human breast cancer cells (ATCC® Number: HTB-20; Lot number: 63087043) were cultured in a DMEM medium containing 10% FBS at 37° C. and 5% CO₂.

Cells were collected and re-suspended in a serum-free DMEM medium, 1×10⁸/mL. The medium was added with and mixed with Matrige at a volume ratio of 1:1 and then placed on ice for use.

Fifty-five nude mice were injected subcutaneously with BT-474 cells, 1×10⁷ cells per mouse, at the right flank. These mice were given intramuscular injections of estrogens one week prior to tumor cell injection till the end of the test, three times a week (every Monday, Wednesday, and Friday), to keep growth of the estrogen-dependent tumor.

When tumor volume reached 100-150 mm³, 36 mice were randomly allocated into 6 groups with 6 mice in each group. Mice were respectively treated, twice a week, through intraperitoneal injection with PBS, IMM01 (3.0 mg/kg), Herceptin (5.0 mg/kg), IMM29 (5.0 mg/kg), IMM2909 (6.0 mg/kg), and IMM01+IMM29 (3.0 mg/kg+5.0 mg/kg), for 3 weeks. Totally six treatments were given. The day upon first dosing was defined as Day 0. Tumor volume and body weight were measured twice a week.

During treatments, if a mouse lost 15% or more of body weight, drug administration would be stopped until the weight loss became 10% or less. Animals were sacrificed when the average tumor volume in any group exceeded 2000 mm³ or the experiment was completed.

The tumor volume (V) was calculated as (length×width2)/2. Tumor growth inhibition rate (TGI) was calculated by the formula: Tumor growth inhibition rate=(1−tumor volume change in administration group/tumor volume change in control group)×100%. Dunnett's multi-comparison test was used to calculate group differences.

TABLE 1
Anti-tumor effect of IMM2902 and other antibodies

| | | | Dose | | |
|---|---|---|---|---|---|
| Group | Drug | Animal# | (mg/kg) | Treatment | TGI* |
| 1 | PBS | 6 | N/A | i.p, b.i.w × 3 | |
| 2 | IMM01 | 6 | 3.0 | i.p, b.i.w × 3 | 25.46% |
| 3 | Herceptin | 6 | 5.0 | i.p, b.i.w × 3 | 86.95% |
| 4 | IMM29 | 6 | 5.0 | i.p, b.i.w × 3 | 104.23% |
| 5 | IMM2902 | 6 | 6.0 | i.p, b.i.w × 3 | 115.28% |
| 6 | IMM01 + | 6 | 3.0 + 5.0 | i.p, b.i.w × 3 | 110.88% |
| | IMM29 | | | | |

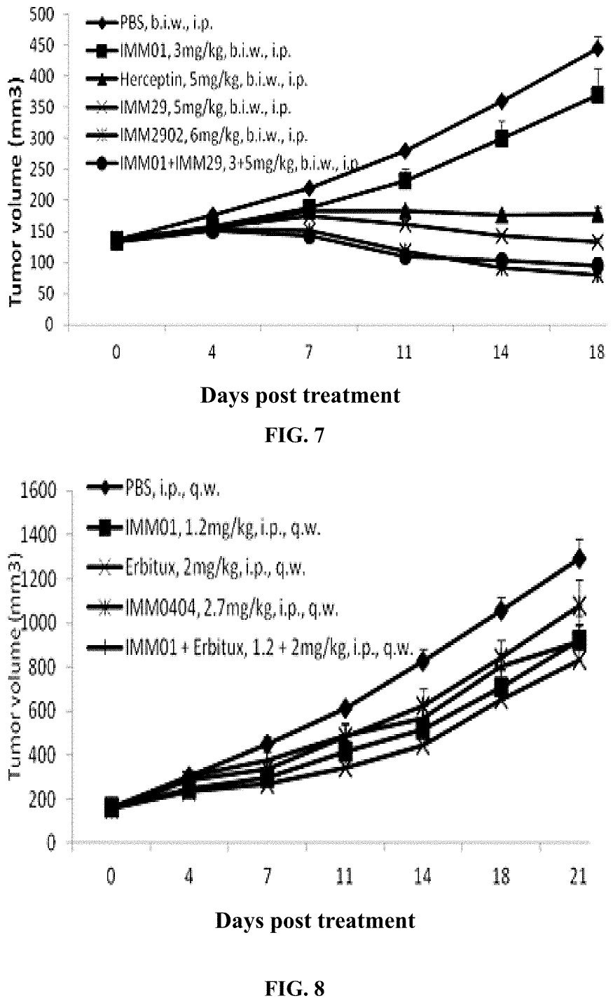

Group 5 had a tumor growth inhibition rate (TGI) of 115.28%, higher than those of other groups, as shown in Table 1 above and FIG. 7, suggesting IMM2902's better efficacy compared to the single antigen targeting antibodies. Especially, the TGI in group 5 was even higher than that of the group with combined use of IMM01 and IMM29.

### Example 8. IMM0404's Anti-Tumor Activity in HT-29 or NCl-H1975 Xenograft Model

8.1 HT-29 Xenograft Model

HT-29 human colon cancer cells were cultured in the McCoy's 5A medium containing 10% FBS at 37° C. and 5% CO₂.

Cells at the logarithmic phase were collected and re-suspended in 1×PBS. The suspension was added with and mixed with Matrige at a volume ratio of 1:1, and the mixture contained 3×10⁷ cells per mL.

Forty mice were injected subcutaneously with HT-29 cells, 3×10⁶ cells per mouse, at the right flank. When tumor volume reached 100-200 mm³, these animals were randomly allocated into 5 groups with 8 mice in each group. Mice were respectively treated, once per week, through intraperitoneal injection with PBS, IMM01 (1.2 mg/kg), Erbitux (2.0 mg/kg), IMM0404 (2.7 mg/kg), and IMM01+Erbitux (1.2 mg/kg+2.0 mg/kg), for 4 weeks. Totally four treatments were given. The day upon first dosing was defined as Day 0. Tumor volume and body weight were measured twice a week.

The tumor volume (V) was calculated as (length×width²)/2. Tumor growth inhibition rate (TGI) was calculated by the formula: Tumor growth inhibition rate=(1−tumor volume change in administration group/tumor volume change in control group)×100%. The student test was used to calculate group differences.

TABLE 2
Anti-tumor effect of IMM0404 and other antibodies

| | | | Dose | | |
|---|---|---|---|---|---|
| Group | Drug | Animal# | (mg/kg) | Treatment | TGI* |
| 1 | PBS | 8 | N/A | i.p, q.w. × 4 | |
| 2 | IMM01 | 8 | 1.2 | i.p, q.w. × 4 | 32.79% |
| 3 | Eribitux | 8 | 2.0 | i.p, q.w. × 4 | 40.00% |
| 4 | IMM0404 | 8 | 2.7 | i.p, q.w. × 4 | 18.48% |
| 5 | IMM01 + | 8 | 1.2 + 2.0 | i.p, q.w. × 4 | 33.04% |
| | Eribitux | | | | |

It can be seen from Table 2 and FIG. 8 that IMM0404 did not show better anti-tumor activity than other proteins in this xenograft model.

8.2 NCI-H1975 Xenograft Model

NCI-H1975 non-small cell lung cancer cells were cultured in the RPMI-1640 medium containing 10% FBS (GIBCO, US) at 37° C. and 5% CO₂.

Cells at the logarithmic phase were collected and re-suspended in 1×PBS, 1×10⁷ cells per mL.

Forty SCID mice were injected subcutaneously with NCI-H1975 cells, 1×10⁶ cells per mouse, at the right flank. When tumor volume reached 100-200 mm³, these animals were randomly allocated into 5 groups with 8 mice in each group. Mice were respectively treated, once per week, through intraperitoneal injection with PBS, IMM01 (2.7 mg/kg), Erbitux (5.0 mg/kg), IMM0404 (6.0 mg/kg), and IMM01+Erbitux (2.7 mg/kg+5.0 mg/kg), for 3 weeks. Totally three treatments were given. The day upon first dosing was defined as Day 0. Tumor volume and body weight were measured twice a week.

The tumor volume (V) was calculated as (length×width²)/2. Tumor growth inhibition rate (TGI) was calculated by the formula: Tumor growth inhibition rate=(1−tumor volume change in administration group/tumor volume change in control group)×100%. The student test was used to calculate group differences.

TABLE 3
Anti-tumor effect of IMM0404 and other antibodies

| | | | Dose | | |
|---|---|---|---|---|---|
| Group | Drug | Animal# | (mg/kg) | Treatment | TGI* |
| 1 | PBS | 8 | N/A | i.p, q.w. × 3 | |
| 2 | IMM01 | 8 | 2.7 | i.p, q.w. × 3 | 49.49% |
| 3 | Eribitux | 8 | 5.0 | i.p, q.w. × 3 | 85.69% |
| 4 | IMM0404 | 8 | 6.0 | i.p, q.w. × 3 | 68.77% |
| 5 | IMM01 + | 8 | 2.7 + 5.0 | i.p, q.w. × 3 | 76.03% |
| | Eribitux | | | | |

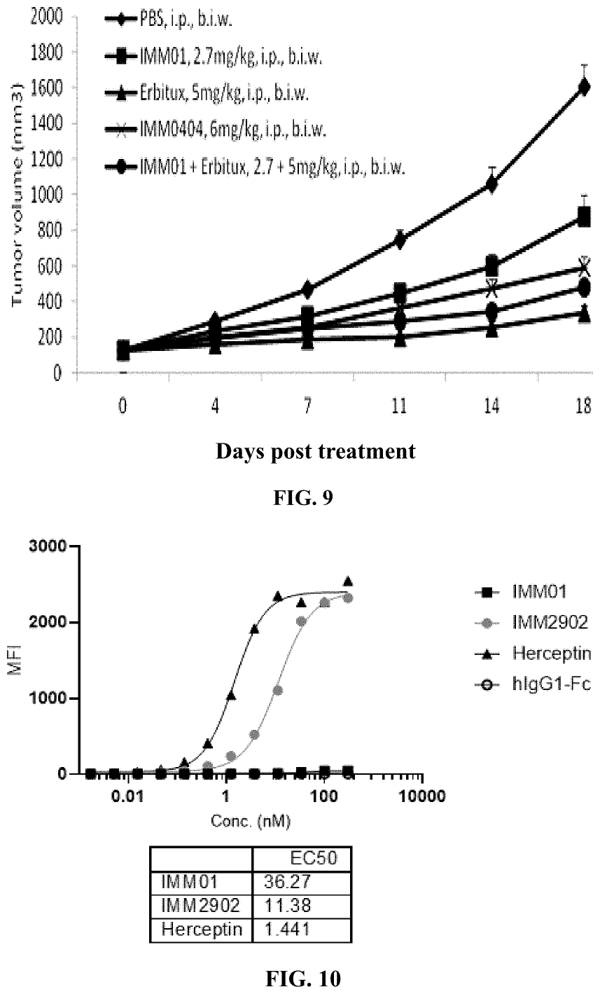

It can be seen from Table 3 and FIG. 9 that IMM0404's anti-tumor activity was better than IMM01, but interior to Eribitux and IMM01+Eribitux.

The data in this Example suggested that the bispecific antibodies do not necessarily show superior efficacy compared to the single antigen targeting antibodies.

### Example 9. Exemplary Recombinant Fusion Protein Bound CD47⁺HER2⁺Tumor Cells

Briefly, 5×10⁵/ml NCl-N87 cells (a gastric cancer cell line, Cat #TCHu130, Cell bank of Chinese Academy of Sciences) in 50 μl 0.5% BSA-PBS were seeded onto 96-well plates. The plates were added with 50 μl serially diluted IMM01, Herceptin®, IMM2902 and hIgG1-Fc, respectively, and incubated for 45 minutes at 4° C. The plates were washed using 0.5% BSA-PBS to remove unbound proteins, added with the secondary antibody FITC anti-human IgG Fc (1:500), and detected for the FITC fluorescence signals by flow cytometry.

The assay was also performed with 5×10⁵/ml SKO-V3 cells (an ovarian cancer cell line, Cat #TCHu185, Cell bank of Chinese Academy of Sciences) in 50 μl 0.5% BSA-PBS, following the protocol above.

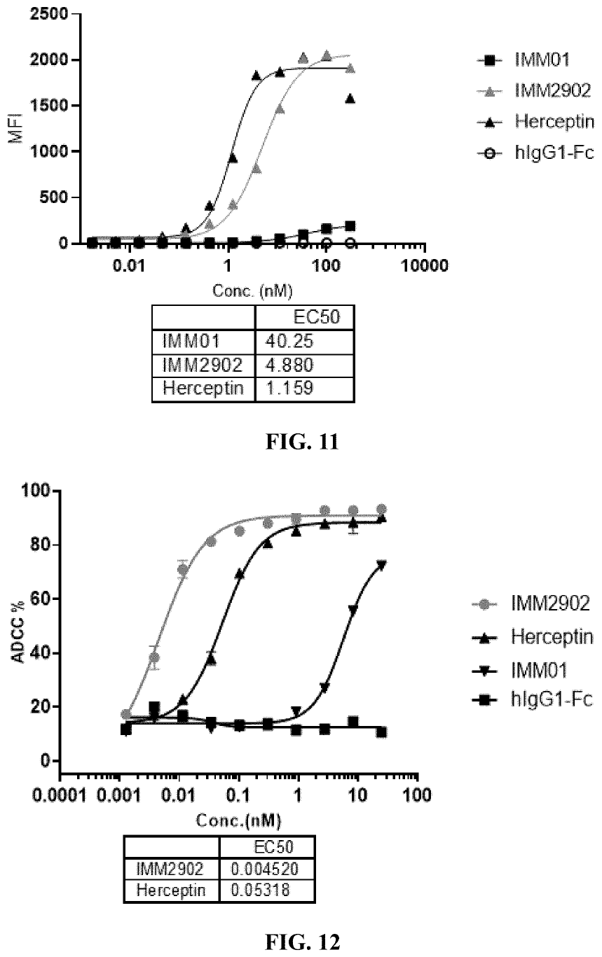

As shown in FIG. 10 and FIG. 11, IMM2902 bound to CD47⁺HER2⁺tumor cells, namely the NCl-N87 cells and SK-OV-3 cells, with EC₅₀ of 11.38 nM and 4.88 nM, respectively.

### Example 10. Exemplary Recombinant Fusion Protein Induced Antibody-Dependent Cell Mediated Cytotoxicity (ADCC) Against CD47⁺HER2⁺Tumor Cells

FcR-TANK cells (ImmuneOnco-engineered cell line, NK cell line overexpress Fc gamma RIIIA), as the effector cells, in 100 μl medium at the density of 5×10⁵/ml, were mixed respectively with NCl-N87 cells and SK-OV-3 cells, as the target cells, in 50 μl medium at 5×10⁵/ml, in 96-well plates. Then, the cell mixtures were added and incubated with 50 μl serially diluted IMM2902, Herceptin®, IMM01 and hIgG1-Fc, respectively, at 37° C. with 5% CO₂ overnight. The plates were added with 20 μl CCK-8 and incubated at 37° C. with 5% CO₂ for 2 hours, and determined for absorbance at 450 nm. The assay was done in duplicate.

ADCC % was calculated using the following formula, wherein OD450(Sample), OD450(FcR-TANK) and OD450(Target cell+FcR-TANK) referred to OD450 value of the plate well with the test article, OD450 value of the well with FcR-TANK cells alone, and OD450 value of the well with target cells and FcR-TANK cells alone, respectively.

$$ADCC \% = \frac{{\overline{OD 450}}_{( Target cell + FcR - TANK )} - {OD 450}_{( Sample )}}{{\overline{OD 450}}_{( Target cell + FcR - TANK )} - {\overline{OD 450}}_{( FcR - TANK )}} \times 100 \%$$

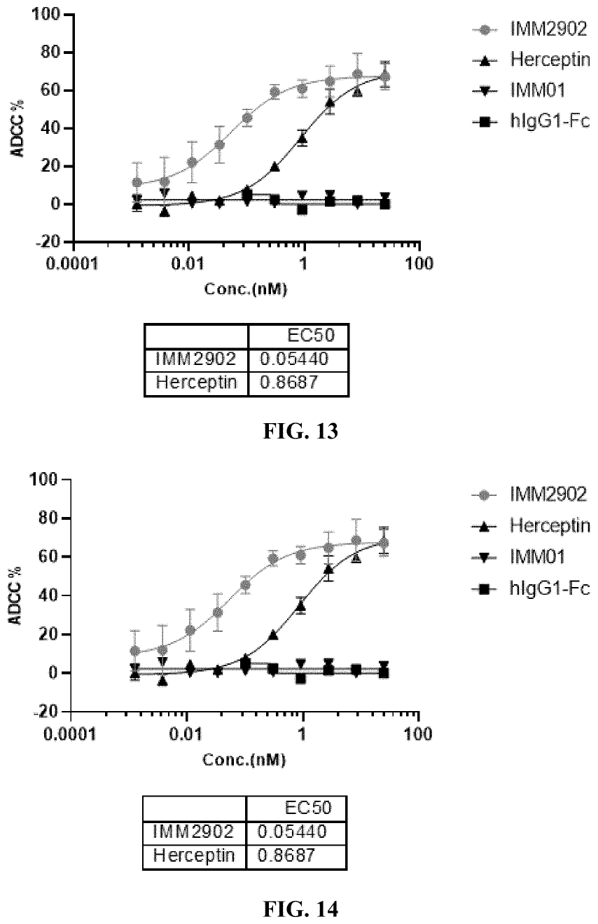

The results were shown in FIG. 12 and FIG. 13. The ADCC induced by IMM2902 against the CD47⁺HER2⁺tumor cells was about 5-15 fold higher than that induced by Herceptin®.

The assay was also performed on the NCl-N87 cells as the target cells and FcR_158V-TANK or FcR_158F-TANK cells as the effector cells, following the protocol above. The FcR_158V-TANK cells expressed on their surfaces Fc gamma RIIIA having 158V mutation while the FcR_158F-TANK cells expressed Fc gamma RIIIA with 158F mutation.

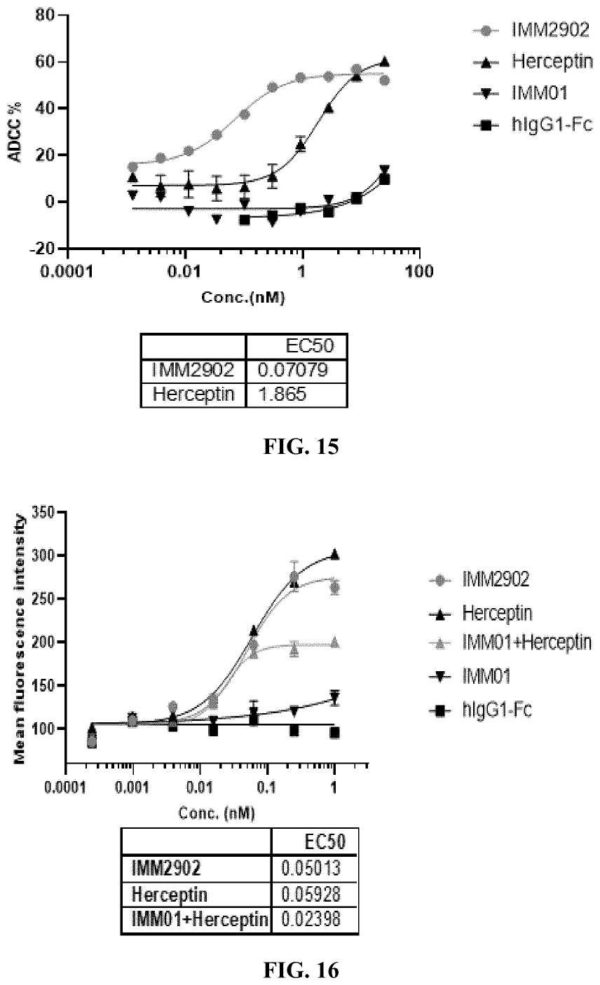

The results were shown in FIG. 14 and FIG. 15. Similarly, IMM2902 induced much higher ADCC than Herceptin®.

### Example 11. Recombinant Fusion Protein Induced Antibody-Dependent Cellular Phagocytosis (ADCP) Against CD47⁺HER2⁺Tumor Cells

THP-1 cells (macrophages) were centrifuged at 1000 rpm for 5 min, collected, washed with the 1640 complete culture medium (1640+10% FBS+1% PS), and cultured at the density of 4×10⁵/ml with 200 ng/ml PMA. Then, 100 μl THP-1 cells were seeded onto each well of 96-well plates, and cultured in a cell incubator for 48 hours.

CFSE at 1 mM was 1:250 diluted into the cell culture with SKO-V3 cells at 1×10⁶/ml to label the tumor cells for 30 min in the cell incubator. The CFSE-labeled SKO-V3 cells were washed with the culture medium and adjusted to the cell density of 1×10⁶/ml.

The supernatants were removed from the plates where the THP-1 cells were cultured. The plates were washed with 100 μl PBS, and added with 100 μl serially diluted IMM2902, Herceptin®, IMM01+Herceptin®, IMM01 or hIgG1-Fc (4-fold dilution starting at 2 nM), and 100 CFSE-labeled SKO-V3, and incubated in the cell incubator for 2 hours.

The plates were washed with 200 μl PBS for three times, incubated with 50 μl trypsin (1:5 dilution in PBS) for 3 min, added with 150 μl PBS, and subject to a flow cytometer to detect fluorescence signals from the THP-1 cells.

As can be seen from FIG. 16, IMM2902 induced phagocytosis of SKO-V3 cells by THP-1 cells, at comparable activity to Herceptin®.

### Example 12. Recombinant Fusion Protein Showed Potent In Vivo Anti-Tumor Activity

SNU-1 cells (a gastric cancer cell line, Cell bank of Chinese Academy of Sciences, Cat #TCHu230) were cultured in RPMI1640 medium with 10% FBS and 1% Penicillin-Streptomycin in a cell incubator at 37° C. and 5% CO₂.

Cells were collected and re-suspended in PBS. PBS, 100 μl in volume, containing 10×10⁶ SNU-1 cells, was added and mixed with 100 μl Matrige.

CB17-SCID female mice, 6-8 weeks old, were injected subcutaneously with the SNU-1 cells as prepared above at the right axilla.

When tumor sizes reached about 142 mm³, the mice were randomly allocated into 5 groups with 6 mice in each group, and this day was designated as Day 0. Mice were intraperitoneally administered with DPBS, Inetetamab (a commercially available anti-HER2 antibody), and IMM2902 respectively following the dosing regime in Table 4.

TABLE 4
Dosing regime

| | | Dose | Dosing volume | |
|---|---|---|---|---|
| Group | Drug | (mg/kg) | (μl/g body weight) | Treatment |
| 1 | DPBS | — | 10 | IP, BIW × 4 W |
| 2 | Inetetamab | 8 | 10 | IP, BIW × 4 W |
| 3 | IMM2902 | 2 | 10 | IP, BIW × 4 W |
| 4 | IMM2902 | 6 | 10 | IP, BIW × 4 W |
| 5 | IMM2902 | 18 | 10 | IP, BIW × 4 W |

The mice were observed for their daily physical activity, and measured for body weights and tumor sizes twice a week. The tumor volume (V) was calculated as (length×width²)/2. Tumor growth inhibition rate (TGI) and relative tumor proliferation rate (T/C, %) were calculated by the following formulae.

Tumor growth inhibition (TGI,%)=(1−tumor volume change in drug treatment group/tumor volume change in vehicle control group)×100%.

T/C (%)=RTV in drug treatment group/RTV in vehicle control group×100%,

RTV=Vt/V0, Vt and V0 referred to the average tumor size at Day t and the average tumor size at Day 0, respectively.

Animal deaths were observed on Day 28 and then on, due to tumor growth. The data at Day 28 were analyzed using t-test in SPSS, and group differences were deemed statistically significant when the p-value was 0.05 or lower.

TABLE 5
Anti-tumor efficacy of IMM2902 and Inetetamab

| | average tumor size at Day 28 | T/C | TGI | |
|---|---|---|---|---|
| Group | (mm³)^{a} | (%) | (%) | p-value ^{b} |
| 1 | 1257.91 ± 157.25 | — | — | — |
| 2 | 800.26 ± 126.69 | 63.66 | 41.02 | 0.047 |
| 3 | 655.51 ± 80.95 | 52.44 | 53.92 | 0.007 |
| 4 | 294.85 ± 56.61 | 23.46 | 86.33 | 0.001 |
| 5 | 239.89 ± 57.67 | 19.17 | 91.20 | 0.001 |

^{a}mean ± SEM;
^{b} compared to Group 1

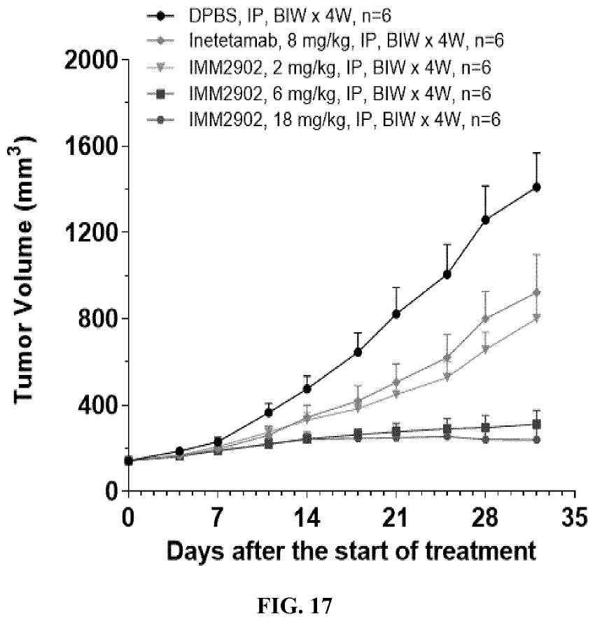

The results were shown in FIG. 17 and Table 5. IMM2902, when administered at the low dose, showed comparable anti-tumor activity to Inetetamab, and when administered at high doses (e.g., 6 mg/kg, 18 mg/kg), showed much better anti-tumor efficacy than Inetetamab.

### Example 13. Recombinant Fusion Protein Showed Higher In Vivo Anti-Tumor Activity than Monospecific Counterparts and their Combination

NCI-N87 cells (a gastric cancer cell line, Cat#TCHu130, Cell bank of Chinese Academy of Sciences) were cultured in RPMI1640 medium with 10% FBS in a cell incubator at 37° C. and 5% CO₂.

Cells at the log phase were collected and re-suspended in RPMI1640 medium. The RPMI1640 medium was mixed with Matrige at 1:1 volume ratio, with the final cell density at 8×10⁷/ml.

CB17-SCID female mice were injected subcutaneously with the NCI-N87 cells as prepared above at the right side of the back, 8×10⁶ cells in 0.1 ml medium per mouse.

When tumor sizes reached about 121 mm³, the mice were randomly allocated into 7 groups with 10 mice in each group, and this day was designated as Day 0. Mice were intraperitoneally administered with PBS, IMM01, Herceptin®, IMM2902 and IMM01+Herceptin®, respectively, following the dosing regime in Table 6.

TABLE 6
Dosing regime

| | | | Dosing | Drug | |
|---|---|---|---|---|---|
| | | | volume | concen- | |
| | | Dose | (μl/g body | tration | |
| Group | Drug | (mg/kg) | weight) | (mg/ml) | Treatment |
| 1 | PBS | NA | 10 | NA | IP, BIW × 4 W |
| 2 | IMM01 | 1.5 | 10 | 0.15 | IP, BIW × 4 W |
| 3 | Herceptin ® | 3 | 10 | 0.3 | IP, BIW × 4 W |
| 4 | IMM2902 | 1 | 10 | 0.1 | IP, BIW × 4 W |
| 5 | IMM2902 | 3.5 | 10 | 0.35 | IP, BIW × 4 W |
| 6 | IMM2902 | 10 | 10 | 1 | IP, BIW × 4 W |
| 7 | IMM01 | 0.5 | 5 | 0.1 | IP, BIW × 4 W |
| | Herceptin ® | 3 | 5 | 0.6 | IP, BIW × 4 W |

The mice were observed for their daily physical activity, and measured for body weights and tumor sizes twice a week. The tumor volume (V) was calculated as (length×width²)/2. Tumor growth inhibition rate (TGI), relative tumor proliferation rate (T/C, %), and animal body weight change (BWC, %) were calculated by the following formulae.

Tumor growth inhibition (TGI,%)=(average tumor size in vehicle control group−average tumor size in drug treatment group)/average tumor size in vehicle control group×100%

T/C (%)=RTV in drug treatment group/RTV in vehicle control group×100%,

wherein RTV (relative tumor volume)=Vt/V0, Vt and V0 referred to the average tumor size at Day t and the average tumor size at Day 0, respectively.

Animal body weight change (BWC,%)=body weight at Day t−body weight at Day 0)/body weight at Day 0×100%

The data were analyzed using the two tailed t-test, and group differences were deemed statistically significant when the p-value was 0.05 or lower, and highly statistically significant when the p-value was 0.01 or lower.

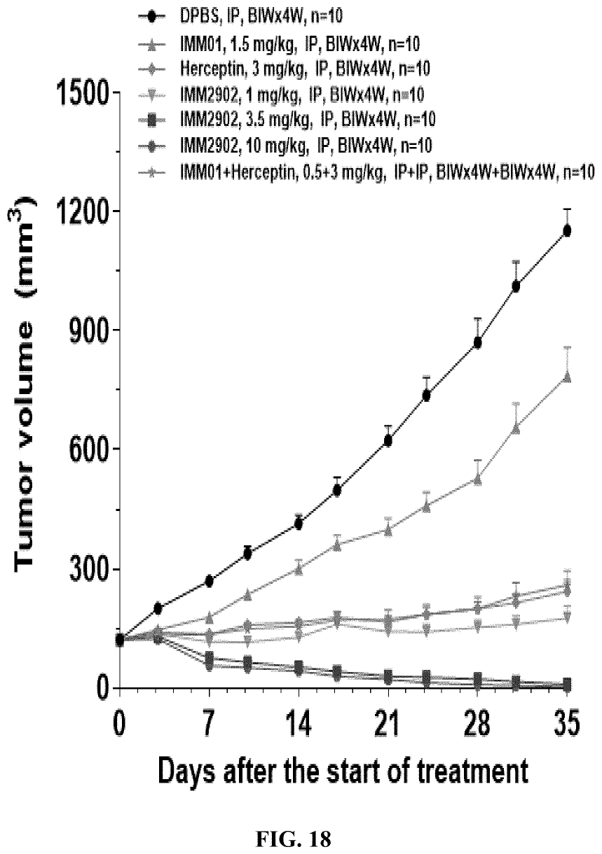

The results were shown in Table 7 and FIG. 18. Basically, no serious animal body weight drop (i.e., weight reduction by 15% or more compared to Day 0), tumor size over 2000 mm³, or adverse effects (including animal disability/blindness) was observed during the test. IMM2902 at the low dose showed comparable anti-tumor activity to Herceptin® and IMM01+Herceptin®, while IMM2902 at high doses provided much higher efficacy than IMM01, Herceptin® and IMM01+Herceptin® combination.

TABLE 7
Anti-tumor efficacy of IMM2902, IMM01, and Herceptin ®

| | | | Tumor size (mm³, Mean ± | | TGI (%)- | |
|---|---|---|---|---|---|---|
| | | Dose | SEM) | | Tumor size | T/C (%) |
| Group | Drug | (mg/kg) | Day 0 | Day 35 | Day 35 | Day 35 |
| 1 | PBS | NA | 121.08 ± 4.2 | 1152.37 ± 53.98 | NA | NA |
| 2 | IMM01 | 1.5 | 121.56 ± 4.31 | 786.78 ± 71.13** | 31.73% | 67.89% |
| 3 | Herceptin ® | 3 | 121.88 ± 4.07 | 242.82 ± 29.62*** | 78.93% | 20.88% |
| 4 | IMM2902 | 1 | 121.61 ± 4.16 | 175.75 ± 29.66*** | 84.75% | 15.42% |
| 5 | IMM2902 | 3.5 | 121.16 ± 3.81 | 8.49 ± 4.41*** | 99.26% | 0.63% |
| 6 | IMM2902 | 10 | 121.07 ± 3.75 | 2.12 ± 2.12*** | 99.82% | 0.21% |
| 7 | IMM01 | 0.5 | 120.76 ± 3.6 | 258.47 ± 36.92*** | 77.57% | 22.56% |
| | Herceptin ® | 3 | | | | |

While the invention has been described above in connection with one or more embodiments, it should be understood that the disclosure is not limited to those embodiments, and the description is intended to cover all alternatives, modifications, and equivalents, as may be included within the spirit and scope of the appended claims. All referenced cited herein are further incorporated by reference in their entirety.

Sequences in the present application are summarized below.

First extracellular Ig-like domain of SIRPalpha
SEQ ID NO: 1
GAGGAGGAGC TGCAGGTGAT TCAGCCTGAC AAGTCCGTAT CAGTTGCAGC TGGAGAGTCG GCCATTCTGC ACTGCACTGT
GACCTCCCTG ATCCCTGTGG GGCCCATCCA GTGGTTCAGA GGAGCTGGAC CAGCCCGGGA ATTAATCTAC AATCAAAAAG
AAGGCCACTT CCCCCGGGTA ACAACTGTTT CAGAGTCCAC AAAGAGAGAA AACATGGACT TTTCCATCAG CATCAGTGCC
ATCACCCCAG CAGATGCCGG CACCTACTAC TGTGTGAAGT TCCGGAAAGG GAGCCCTGAC ACGGAGTTTA AGTCTGGAGC
AGGCACTGAG CTGTCTGTGC GTGCCAAACC CTCTGCCCCC GTGGTATCGG GCCCT 375
First extracellular Ig-like domain of SIRPalpha
SEQ ID NO: 2
EEELQVIQPD KSVSVAAGES AILHCTVTSL IPVGPIQWFR GAGPARELIY NQKEGHFPRV TTVSESTKRE NMDFSISISA
ITPADAGTYY CVKFRKGSPD TEFKSGAGTE LSVRAKPSAP WSGP 125
Linker
SEQ ID NO: 3
GGCGG CGGTGGGAGC GGCGGCGGTG GGAGCGGCGG CGGGGGCTCG 45
Linker
SEQ ID NO: 4
GGGGSGGGGS GGGGS 15
Heavy chain of anti-HER2 antibody
SEQ ID NO: 5
GAGGTGCAGC TGGTCGAGAG CGGCGGGGGC CTCGTGCAGC CGGGCGGGTC GCTGCGGCTG AGCTGCGCCG CGAGCGGGTT
CAACATCAAG GACACCTACA TCCACTGGGT GCGCCAGGCC CCCGGCAAGG GCCTCGAGTG GGTCGCCCGG ATCTACCCCA
CGAACGGGTA CACCCGCTAC GCCGACAGCG TGAAGGGCCG GTTCACCATC AGCGCGGACA CCTCGAAGAA CACGGCCTAC
CTGCAGATGA ACAGCCTGCG CGCCGAGGAC ACCGCCGTGT ACTACTGCAG CCGGTGGGGC GGCGACGGGT TCTACGCCAT
GGACTACTGG GGGCAGGGCA CCCTCGTCAC CGTGAGCAGC GCTAGCACCA AGGGCCCATC GGTCTTCCCC CTGGCACCCT
CCTCCAAGAG CACCTCTGGG GGCACAGCGG CCCTGGGCTG CCTGGTCAAG GACTACTTCC CCGAACCGGT GACGGTGTCG
TGGAACTCAG GCGCCCTGAC CAGCGGCGTG CACACCTTCC CGGCTGTCCT ACAGTCCTCA GGACTCTACT CCCTCAGCAG
CGTGGTGACC GTGCCCTCCA GCAGCTTGGG CACCCAGACC TACATCTGCA ACGTGAATCA CAAGCCCAGC AACACCAAGG
TGGACAAGAG AGTTGAGCCC AAATCTTGTG ACAAAACTCA CACATGCCCA CCGTGCCCAG CACCTGAACT CCTGGGGGGA
CCGTCAGTCT TCCTCTTCCC CCCAAAACCC AAGGACACCC TCATGATCTC CCGGACCCCT GAGGTCACAT GCGTGGTGGT
GGACGTGAGC CACGAAGACC CTGAGGTCAA GTTCAACTGG TATGTGGACG GCGTGGAGGT GCATAATGCC AAGACAAAGC
CGCGGGAGGA GCAGTACAAC GCCACGTACC GTGTGGTCAG CGTCCTCACC GTCCTGCACC AAGACTGGCT GAATGGCAAG
GAGTACAAGT GCAAGGTCTC CAACAAAGCC CTCCCAGCCC CCATCGCCGC AACCATCTCC AAAGCCAAAG GGCAGCCCCG
AGAACCACAG GTGTACACCC TGCCCCCATC CCGGGAGGAG ATGACCAAGA ACCAAGTCAG CCTGACCTGC CTGGTCAAAG
GCTTCTATCC CAGCGACATC GCCGTGGAGT GGGAGAGCAA TGGGCAGCCG GAGAACAACT ACAAGACCAC GCCTCCCGTG
CTGGACTCCG ACGGCTCCTT CTTCCTCTAT TCCAAGCTCA CCGTGGACAA GAGCAGGTGG CAGCAGGGGA ACGTCTTCTC
ATGCTCCGTG ATGCATGAGG CTCTGCACAA CCACTACACG CAGAAGAGCC TCTCCCTGTC TCCGGGCAAA TGA 1353
Heavy chain of anti-HER2 antibody
SEQ ID NO: 6
EVQLVESGGG LVQPGGSLRL SCAASGFNIK DTYIHWVRQA PGKGLEWVAR IYPTNGYTRY ADSVKGRFTI SADTSKNTAY
LQMNSLRAED TAVYYCSRWG GDGFYAMDYW GQGTLVTVSS ASTKGPSVFP LAPSSKSTSG GTAALGCLVK DYFPEPVTVS
WNSGALTSGV HTFPAVLQSS GLYSLSSVVT VPSSSLGTQT YICNVNHKPS NTKVDKRVEP KSCDKTHTCP PCPAPELLGG
PSVFLFPPKP KDTLMISRTP EVTCVVVDVS HEDPEVKFNW YVDGVEVHNA KTKPREEQYN ATYRVVSVLT VLHQDWLNGK
EYKCKVSNKA LPAPIAATIS KAKGQPREPQ VYTLPPSREE MTKNQVSLTC LVKGFYPSDI AVEWESNGQP ENNYKTTPPV
LDSDGSFFLY SKLTVDKSRW QQGNVFSCSV MHEALHNHYT QKSLSLSPGK 450
Light chain of anti-HER2 antibody
SEQ ID NO: 7
GACATCCAGA TGACCCAGAG CCCGTCGAGC CTGAGCGCCA GCGTGGGCGA CCGGGTCACG ATCACCTGCC GCGCGAGCCA
GGACGTGAAC ACCGCCGTGG CCTGGTACCA GCAGAAGCCC GGGAAGGCCC CCAAGCTCCT GATCTACTCG GCGAGCTTCC
TGTACAGCGG CGTCCCCAGC CGGTTCAGCG GGTCGCGCAG CGGCACCGAC TTCACGCTCA CCATCAGCAG CCTGCAGCCG
GAGGACTTCG CCACCTACTA CTGCCAGCAG CACTACACCA CGCCCCCCAC CTTCGGGCAG GGCACCAAGG TGGAGATCAA
GCGAACTGTG GCTGCACCAT CTGTCTTCAT CTTCCCGCCA TCTGATGAGC AGTTGAAATC TGGAACTGCC TCTGTTGTGT
GCCTGCTGAA TAACTTCTAT CCCAGAGAGG CCAAAGTACA GTGGAAGGTG GATAACGCCC TCCAATCGGG TAACTCCCAG
GAGAGTGTCA CAGAGCAGGA CAGCAAGGAC AGCACCTACA GCCTCAGCAG CACCCTGACG CTGAGCAAAG CAGACTACGA
GAAACACAAA GTCTACGCCT GCGAAGTCAC CCATCAGGGC CTGAGCTCGC CCGTCACAAA GAGCTTCAAC AGGGGAGAGT
GTTAG 645
Light chain of anti-HER2 antibody
SEQ ID NO: 8
DIQMTQSPSS LSASVGDRVT ITCRASQDVN TAVAWYQQKP GKAPKLLIYS ASFLYSGVPS RFSGSRSGTD FTLTISSLQP
EDFATYYCQQ HYTTPPTFGQ GTKVEIKRTV AAPSVFIFPP SDEQLKSGTA SVVCLLNNFY PREAKVQWKV DNALQSGNSQ
ESVTEQDSKD STYSLSSTLT LSKADYEKHK VYACEVTHQG LSSPVTKSFN RGEC 214
SIRP alpha Dl-Linker-anti-HER2 heavy chain
SEQ ID NO: 9
GAGGAGGAGC TGCAGGTGAT TCAGCCTGAC AAGTCCGTAT CAGTTGCAGC TGGAGAGTCG GCCATTCTGC ACTGCACTGT
GACCTCCCTG ATCCCTGTGG GGCCCATCCA GTGGTTCAGA GGAGCTGGAC CAGCCCGGGA ATTAATCTAC AATCAAAAAG
AAGGCCACTT CCCCCGGGTA ACAACTGTTT CAGAGTCCAC AAAGAGAGAA AACATGGACT TTTCCATCAG CATCAGTGCC
ATCACCCCAG CAGATGCCGG CACCTACTAC TGTGTGAAGT TCCGGAAAGG GAGCCCTGAC ACGGAGTTTA AGTCTGGAGC
AGGCACTGAG CTGTCTGTGC GTGCCAAACC CTCTGCCCCC GTGGTATCGG GCCCTGGCGG CGGTGGGAGC GGCGGCGGTG
GGAGCGGCGG CGGGGGCTCG GAGGTGCAGC TGGTCGAGAG CGGCGGGGGC CTCGTGCAGC CGGGCGGGTC GCTGCGGCTG
AGCTGCGCCG CGAGCGGGTT CAACATCAAG GACACCTACA TCCACTGGGT GCGCCAGGCC CCCGGCAAGG GCCTCGAGTG
GGTCGCCCGG ATCTACCCCA CGAACGGGTA CACCCGCTAC GCCGACAGCG TGAAGGGCCG GTTCACCATC AGCGCGGACA
CCTCGAAGAA CACGGCCTAC CTGCAGATGA ACAGCCTGCG CGCCGAGGAC ACCGCCGTGT ACTACTGCAG CCGGTGGGGC
GGCGACGGGT TCTACGCCAT GGACTACTGG GGGCAGGGCA CCCTCGTCAC CGTGAGCAGC GCTAGCACCA AGGGCCCATC
GGTCTTCCCC CTGGCACCCT CCTCCAAGAG CACCTCTGGG GGCACAGCGG CCCTGGGCTG CCTGGTCAAG GACTACTTCC
CCGAACCGGT GACGGTGTCG TGGAACTCAG GCGCCCTGAC CAGCGGCGTG CACACCTTCC CGGCTGTCCT ACAGTCCTCA
GGACTCTACT CCCTCAGCAG CGTGGTGACC GTGCCCTCCA GCAGCTTGGG CACCCAGACC TACATCTGCA ACGTGAATCA
CAAGCCCAGC AACACCAAGG TGGACAAGAG AGTTGAGCCC AAATCTTGTG ACAAAACTCA CACATGCCCA CCGTGCCCAG
CACCTGAACT CCTGGGGGGA CCGTCAGTCT TCCTCTTCCC CCCAAAACCC AAGGACACCC TCATGATCTC CCGGACCCCT
GAGGTCACAT GCGTGGTGGT GGACGTGAGC CACGAAGACC CTGAGGTCAA GTTCAACTGG TATGTGGACG GCGTGGAGGT
GCATAATGCC AAGACAAAGC CGCGGGAGGA GCAGTACAAC GCCACGTACC GTGTGGTCAG CGTCCTCACC GTCCTGCACC
AAGACTGGCT GAATGGCAAG GAGTACAAGT GCAAGGTCTC CAACAAAGCC CTCCCAGCCC CCATCGCCGC AACCATCTCC
AAAGCCAAAG GGCAGCCCCG AGAACCACAG GTGTACACCC TGCCCCCATC CCGGGAGGAG ATGACCAAGA ACCAAGTCAG
CCTGACCTGC CTGGTCAAAG GCTTCTATCC CAGCGACATC GCCGTGGAGT GGGAGAGCAA TGGGCAGCCG GAGAACAACT
ACAAGACCAC GCCTCCCGTG CTGGACTCCG ACGGCTCCTT CTTCCTCTAT TCCAAGCTCA CCGTGGACAA GAGCAGGTGG
CAGCAGGGGA ACGTCTTCTC ATGCTCCGTG ATGCATGAGG CTCTGCACAA CCACTACACG CAGAAGAGCC TCTCCCTGTC
TCCGGGCAAA TGA 1773
SIRP alpha D1-Linker-anti-HER2 heavy chain
SEQ ID NO: 10
EEELQVIQPD KSVSVAAGES AILHCTVTSL IPVGPIQWFR GAGPARELIY NQKEGHFPRV TTVSESTKRE NMDFSISISA
ITPADAGTYY CVKFRKGSPD TEFKSGAGTE LSVRAKPSAP VVSGPGGGGS GGGGSGGGGS EVQLVESGGG LVQPGGSLRL
SCAASGFNIK DTYIHWVRQA PGKGLEWVAR IYPTNGYTRY ADSVKGRFTI SADTSKNTAY LQMNSLRAED TAVYYCSRWG
GDGFYAMDYW GQGTLVTVSS ASTKGPSVFP LAPSSKSTSG GTAALGCLVK DYFPEPVTVS WNSGALTSGV HTFPAVLQSS
GLYSLSSVVT VPSSSLGTQT YICNVNHKPS NTKVDKRVEP KSCDKTHTCP PCPAPELLGG PSVFLFPPKP KDTLMISRTP
EVTCVVVDVS HEDPEVKFNW YVDGVEVHNA KTKPREEQYN ATYRVVSVLT VLHQDWLNGK EYKCKVSNKA LPAPIAATIS
KAKGQPREPQ VYTLPPSREE MTKNQVSLTC LVKGFYPSDI AVEWESNGQP ENNYKTTPPV LDSDGSFFLY SKLTVDKSRW
QQGNVFSCSV MHEALHNHYT QKSLSLSPGK 590
SIRP alpha Dl-Linker-anti-HER2 light chain
SEQ ID NO: 11
GAGGAGGAGC TGCAGGTGAT TCAGCCTGAC AAGTCCGTAT CAGTTGCAGC TGGAGAGTCG GCCATTCTGC ACTGCACTGT
GACCTCCCTG ATCCCTGTGG GGCCCATCCA GTGGTTCAGA GGAGCTGGAC CAGCCCGGGA ATTAATCTAC AATCAAAAAG
AAGGCCACTT CCCCCGGGTA ACAACTGTTT CAGAGTCCAC AAAGAGAGAA AACATGGACT TTTCCATCAG CATCAGTGCC
ATCACCCCAG CAGATGCCGG CACCTACTAC TGTGTGAAGT TCCGGAAAGG GAGCCCTGAC ACGGAGTTTA AGTCTGGAGC
AGGCACTGAG CTGTCTGTGC GTGCCAAACC CTCTGCCCCC GTGGTATCGG GCCCTGGCGG CGGTGGGAGC GGCGGCGGTG
GGAGCGGCGG CGGGGGCTCG GACATCCAGA TGACCCAGAG CCCGTCGAGC CTGAGCGCCA GCGTGGGCGA CCGGGTCACG
ATCACCTGCC GCGCGAGCCA GGACGTGAAC ACCGCCGTGG CCTGGTACCA GCAGAAGCCC GGGAAGGCCC CCAAGCTCCT
GATCTACTCG GCGAGCTTCC TGTACAGCGG CGTCCCCAGC CGGTTCAGCG GGTCGCGCAG CGGCACCGAC TTCACGCTCA
CCATCAGCAG CCTGCAGCCG GAGGACTTCG CCACCTACTA CTGCCAGCAG CACTACACCA CGCCCCCCAC CTTCGGGCAG
GGCACCAAGG TGGAGATCAA GCGAACTGTG GCTGCACCAT CTGTCTTCAT CTTCCCGCCA TCTGATGAGC AGTTGAAATC
TGGAACTGCC TCTGTTGTGT GCCTGCTGAA TAACTTCTAT CCCAGAGAGG CCAAAGTACA GTGGAAGGTG GATAACGCCC
TCCAATCGGG TAACTCCCAG GAGAGTGTCA CAGAGCAGGA CAGCAAGGAC AGCACCTACA GCCTCAGCAG CACCCTGACG
CTGAGCAAAG CAGACTACGA GAAACACAAA GTCTACGCCT GCGAAGTCAC CCATCAGGGC CTGAGCTCGC CCGTCACAAA
GAGCTTCAAC AGGGGAGAGT GTTAG 1065
SIRP alpha D1-Linker-anti-HER2 light chain
SEQ ID NO: 12
EEELQVIQPD KSVSVAAGES AILHCTVTSL IPVGPIQWFR GAGPARELIY NQKEGHFPRV TTVSESTKRE NMDFSISISA
ITPADAGTYY CVKFRKGSPD TEFKSGAGTE LSVRAKPSAP VVSGPGGGGS GGGGSGGGGS DIQMTQSPSS LSASVGDRVT
ITCRASQDVN TAVAWYQQKP GKAPKLLIYS ASFLYSGVPS RFSGSRSGTD FTLTISSLQP EDFATYYCQQ HYTTPPTFGQ
GTKVEIKRTV AAPSVFIFPP SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC 354
SIRP alpha Dl-Fc
SEQ ID NO: 13
GAGGAGGAGC TGCAGGTGAT TCAGCCTGAC AAGTCCGTAT CAGTTGCAGC TGGAGAGTCG GCCATTCTGC ACTGCACTGT
GACCTCCCTG ATCCCTGTGG GGCCCATCCA GTGGTTCAGA GGAGCTGGAC CAGCCCGGGA ATTAATCTAC AATCAAAAAG
AAGGCCACTT CCCCCGGGTA ACAACTGTTT CAGAGTCCAC AAAGAGAGAA AACATGGACT TTTCCATCAG CATCAGTGCC
ATCACCCCAG CAGATGCCGG CACCTACTAC TGTGTGAAGT TCCGGAAAGG GAGCCCTGAC ACGGAGTTTA AGTCTGGAGC
AGGCACTGAG CTGTCTGTGC GTGCCAAACC CTCTGCCCCC GTGGTATCGG GCCCTGCGGC GAGGGCCACA CCTCAGCACG
AATTCGAGCC CAAATCTTGT GACAAAACTC ACACATGCCC ACCGTGCCCA GCACCTGAAC TCCTGGGGGG ACCGTCAGTC
TTCCTCTTCC CCCCAAAACC CAAGGACACC CTCATGATCT CCCGGACCCC TGAGGTCACA TGCGTGGTGG TGGACGTGAG
CCACGAAGAC CCTGAGGTCA AGTTCAACTG GTACGTGGAC GGCGTGGAGG TGCATAATGC CAAGACAAAG CCGCGGGAGG
AGCAGTACAA CAGCACGTAC CGTGTGGTCA GCGTCCTCAC CGTCCTGCAC CAGGACTGGC TGAATGGCAA GGAGTACAAG
TGCAAGGTCT CCAACAAAGC CCTCCCAGCC CCCATCGAGA AAACCATCTC CAAAGCCAAA GGGCAGCCCC GAGAACCACA
GGTGTACACC CTGCCCCCAT CCCGGGATGA GCTGACCAAG AACCAGGTCA GCCTGACCTG CCTGGTCAAA GGCTTCTATC
CCAGCGACAT CGCCGTGGAG TGGGAGAGCA ATGGGCAGCC GGAGAACAAC TACAAGACCA CGCCTCCCGT GCTGGACTCC
GACGGCTCCT TCTTCCTCTA CAGCAAGCTC ACCGTGGACA AGAGCAGGTG GCAGCAGGGG AACGTCTTCT CATGCTCCGT
GATGCATGAG GCTCTGCACA ACCACTACAC GCAGAAGAGC CTCTCCCTGT CTCCGGGTTG A 1101
SIRP alpha Dl-Fc
SEQ ID NO: 14
EEELQVIQPD KSVSVAAGES AILHCTVTSL IPVGPIQWFR GAGPARELIY NQKEGHFPRV TTVSESTKRE NMDFSISISA
ITPADAGTYY CVKFRKGSPD TEFKSGAGTE LSVRAKPSAP VVSGPAARAT PQHEFEPKSC DKTHTCPPCP APELLGGPSV
FLFPPKPKDT LMISRTPEVT CVVVDVSHED PEVKFNWYVD GVEVHNAKTK PREEQYNSTY RVVSVLTVLH QDWLNGKEYK
CKVSNKALPA PIEKTISKAK GQPREPQVYT LPPSRDELTK NQVSLTCLVK GFYPSDIAVE WESNGQPENN YKTTPPVLDS
DGSFFLYSKL TVDKSRWQQG NVFSCSVMHE ALHNHYTQKS LSLSPG 366
SIRP alpha Dl-Linker-anti-EGFR light chain
SEQ ID NO: 15
GAGGAGGAGC TGCAGGTGAT TCAGCCTGAC AAGTCCGTAT CAGTTGCAGC TGGAGAGTCG GCCATTCTGC ACTGCACTGT
GACCTCCCTG ATCCCTGTGG GGCCCATCCA GTGGTTCAGA GGAGCTGGAC CAGCCCGGGA ATTAATCTAC AATCAAAAAG
AAGGCCACTT CCCCCGGGTA ACAACTGTTT CAGAGTCCAC AAAGAGAGAA AACATGGACT TTTCCATCAG CATCAGTGCC
ATCACCCCAG CAGATGCCGG CACCTACTAC TGTGTGAAGT TCCGGAAAGG GAGCCCTGAC ACGGAGTTTA AGTCTGGAGC
AGGCACTGAG CTGTCTGTGC GTGCCAAACC CTCTGGCGGC GGTGGGAGCG GCGGCGGTGG GAGCGGCGGC GGGGGCTCGC
AGGTGCAGCT GAAGCAGTCA GGACCTGGCC TAGTGCAGCC CTCACAGAGC CTGTCCATCA CCTGCACAGT CTCTGGTTTC
TCATTAACTA ACTATGGTGT ACACTGGGTT CGCCAGTCTC CAGGAAAGGG TCTGGAGTGG CTGGGAGTGA TATGGAGTGG
TGGAAACACA GACTATAATA CACCTTTCAC ATCCAGACTG AGCATCAACA AGGACAATTC CAAGAGCCAA GTTTTCTTTA
AAATGAACAG TCTGCAATCT CAGGACACAG CCATATATTA CTGTGCCAGA GCCCTCACCT ACTATGATTA CGAGTTTGCT
TACTGGGGCC AAGGGACTCT GGTCACTGTC TCTGCAGCTA GCACCAAGGG CCCATCGGTC TTCCCCCTGG CACCCTCCTC
CAAGAGCACC TCTGGGGGCA CAGCGGCCCT GGGCTGCCTG GTCAAGGACT ACTTCCCCGA ACCGGTGACG GTGTCGTGGA
ACTCAGGCGC CCTGACCAGC GGCGTGCACA CCTTCCCGGC TGTCCTACAG TCCTCAGGAC TCTACTCCCT CAGCAGCGTG
GTGACCGTGC CCTCCAGCAG CTTGGGCACC CAGACCTACA TCTGCAACGT GAATCACAAG CCCAGCAACA CCAAGGTGGA
CAAGAGAGTT GAGCCCAAAT CTTGTGACAA AACTCACACA TGCCCACCGT GCCCAGCACC TGAACTCCTG GGGGGACCGT
CAGTCTTCCT CTTCCCCCCA AAACCCAAGG ACACCCTCAT GATCTCCCGG ACCCCTGAGG TCACATGCGT GGTGGTGGAC
GTGAGCCACG AAGACCCTGA GGTCAAGTTC AACTGGTATG TGGACGGCGT GGAGGTGCAT AATGCCAAGA CAAAGCCGCG
GGAGGAGCAG TACAACGCCA CGTACCGTGT GGTCAGCGTC CTCACCGTCC TGCACCAAGA CTGGCTGAAT GGCAAGGAGT
ACAAGTGCAA GGTCTCCAAC AAAGCCCTCC CAGCCCCCAT CGCCGCAACC ATCTCCAAAG CCAAAGGGCA GCCCCGAGAA
CCACAGGTGT ACACCCTGCC CCCATCCCGG GAGGAGATGA CCAAGAACCA AGTCAGCCTG ACCTGCCTGG TCAAAGGCTT
CTATCCCAGC GACATCGCCG TGGAGTGGGA GAGCAATGGG CAGCCGGAGA ACAACTACAA GACCACGCCT CCCGTGCTGG
ACTCCGACGG CTCCTTCTTC CTCTATTCCA AGCTCACCGT GGACAAGAGC AGGTGGCAGC AGGGGAACGT CTTCTCATGC
TCCGTGATGC ATGAGGCTCT GCACAACCAC TACACGCAGA AGAGCCTCTC CCTGTCTCCG GGCAAATGA 1749
SIRP alpha D1-Linker-anti-EGFR light chain
SEQ ID NO: 16
EEELQVIQPD KSVSVAAGES AILHCTVTSL IPVGPIQWFR GAGPARELIY NQKEGHFPRV TTVSESTKRE NMDFSISISA
ITPADAGTYY CVKFRKGSPD TEFKSGAGTE LSVRAKPSGG GGSGGGGSGG GGSQVQLKQS GPGLVQPSQS LSITCTVSGF
SLTNYGVHWV RQSPGKGLEW LGVIWSGGNT DYNTPFTSRL SINKDNSKSQ VFFKMNSLQS QDTAIYYCAR ALTYYDYEFA
YWGQGTLVTV SAASTKGPSV FPLAPSSKST SGGTAALGCL VKDYFPEPVT VSWNSGALTS GVHTFPAVLQ SSGLYSLSSV
VTVPSSSLGT QTYICNVNHK PSNTKVDKRV EPKSCDKTHT CPPCPAPELL GGPSVFLFPP KPKDTLMISR TPEVTCVWD
VSHEDPEVKF NWYVDGVEVH NAKTKPREEQ YNATYRVVSV LTVLHQDWLN GKEYKCKVSN KALPAPIAAT ISKAKGQPRE
PQVYTLPPSR EEMTKNQVSL TCLVKGFYPS DIAVEWESNG QPENNYKTTP PVLDSDGSFF LYSKLTVDKS RWQQGNVFSC
SVMHEALHNH YTQKSLSLSP GK 582
Light chain of anti-EGFR antibody
SEQ ID NO: 17
GACATCTTGC TGACTCAGTC TCCAGTCATC CTGTCTGTGA GTCCAGGAGA AAGAGTCAGT TTCTCCTGCA GGGCCAGTCA
GAGTATTGGC ACAAACATAC ACTGGTATCA GCAAAGAACA AATGGTTCTC CAAGGCTTCT CATAAAGTAT GCTTCTGAGT
CTATCTCTGG GATCCCTTCC AGGTTTAGTG GCAGTGGATC AGGGACAGAT TTTACTCTTA GCATCAACAG TGTGGAGTCT
GAAGATATTG CAGATTATTA CTGTCAACAA AATAATAACT GGCCAACCAC GTTCGGTGCT GGGACCAAGC TGGAGCTGAA
ACGAACTGTG GCTGCACCAT CTGTCTTCAT CTTCCCGCCA TCTGATGAGC AGTTGAAATC TGGAACTGCC TCTGTTGTGT
GCCTGCTGAA TAACTTCTAT CCCAGAGAGG CCAAAGTACA GTGGAAGGTG GATAACGCCC TCCAATCGGG TAACTCCCAG
GAGAGTGTCA CAGAGCAGGA CAGCAAGGAC AGCACCTACA GCCTCAGCAG CACCCTGACG CTGAGCAAAG CAGACTACGA
GAAACACAAA GTCTACGCCT GCGAAGTCAC CCATCAGGGC CTGAGCTCGC CCGTCACAAA GAGCTTCAAC AGGGGAGAGT
GTTAG 645
Light chain of anti-EGFR antibody
SEQ ID NO: 18
DILLTQSPVI LSVSPGERVS FSCRASQSIG TNIHWYQQRT NGSPRLLIKY ASESISGIPS RFSGSGSGTD FTLSINSVES
EDIADYYCQQ NNNWPTTFGA GTKLELKRTV AAPSVFIFPP SDEQLKSGTA SVVCLLNNFY PREAKVQWKV DNALQSGNSQ
ESVTEQDSKD STYSLSSTLT LSKADYEKHK VYACEVTHQG LSSPVTKSFN RGEC 214
signal peptide of mouse IgGl heavy chain
SEQ ID NO: 19
ATGGGATGGT CATGTATCAT CCTTTTTCTG GTAGCAACTG CAACTGGAGT ACATTCA 57
SEQ ID NO: 20
GCCGCCACC
Linker
SEQ ID NO: 21
GGGGS GGGGS GGGGS 15

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method for treating gastric cancer caused by over-expression of CD47 and/or HER2, comprising administering to a patient or a subject a therapeutically effective amount of a pharmaceutical composition comprising a recombinant fusion protein and at least one pharmaceutical carrier;
wherein the recombinant fusion protein comprises an extracellular Ig-like domain of a signal-regulatory protein (SIRP), linked via a linker, to a paratope of an Ig-like anti-HER2 antibody at the N-terminus of a heavy chain or a light chain constituting the paratope;
wherein the recombinant fusion protein is capable of blocking binding of CD47s on cancer cells to SIRPs on surfaces of macrophages, binding to HER2s on cancer cells to inhibit uncontrolled cancer cell growth, and binding to FcRs on NK cells or macrophages.

2. The method for the recombinant fusion protein of claim 1, wherein each paratope of the Ig-like anti-HER2 antibody is linked to an extracellular Ig-like domain of signal-regulatory protein (SIRP) at the N-terminus of the heavy chain constituting that paratope.

3. The method for the recombinant fusion protein of claim 1, wherein each paratope of the Ig-like anti-HER2 antibody is linked to an extracellular Ig-like domain of signal-regulatory protein (SIRP) at the N-terminus of the light chain constituting that paratope.

4. The method for the recombinant fusion protein of claim 1, wherein one paratope of the Ig-like anti-HER2 antibody is linked to an extracellular Ig-like domain of signal-regulatory protein (SIRP) at the N-terminus of the heavy chain constituting that paratope, and the other paratope of the Ig-like anti-HER2 antibody is linked to an extracellular Ig-like domain of signal-regulatory protein (SIRP) at the N-terminus of the light chain constituting that paratope.

5. The method for the recombinant fusion protein of claim 1, wherein the signal-regulatory protein is SIRPα.

6. The method for the recombinant fusion protein of claim 5, wherein the extracellular Ig-like domain of the signal-regulatory protein is first extracellular Ig-like domain of signal-regulatory protein (SIRPαD1).

7. The method for the recombinant fusion protein of claim 1, wherein the linker is a peptide of 10 to 30 amino acid residues.

8. The method for the recombinant fusion protein of claim 7, wherein the linker is -(Gly-Gly-Gly-Gly-Ser)3-.

9. The method for the recombinant fusion protein of claim 1, wherein the Ig-like anti-HER2 antibody comprises two heavy chains each having an amino acid sequence with at least 80%, 85%, 90%, 95%, 98% or 99% identity to SEQ ID NO: 6, and two light chains each having an amino acid sequence with at least 80%, 85%, 90%, 95%, 98% or 99% identity to SEQ ID NO: 8.

10. The method for the recombinant fusion protein of claim 9, wherein each heavy chain has an amino acid sequence of SEQ ID NO: 6.

11. The method for the recombinant fusion protein of claim 9, wherein each light chain has an amino acid sequence of SEQ ID NO: 8.


SEQUENCE LISTING (21 sequences)

SEQ ID NO: 1 - DNA, 375 bp, synthetic construct
  source 1..375: mol_type = other DNA
gaggaggagctgcaggtgattcagcctgacaagtccgtatcagttgcagctggagagtcggccattctgcactgcactgtgacctccctgatccctgtggggcccatccagtggttcagaggagctggaccagcccgggaattaatctacaatcaaaaagaaggccacttcccccgggtaacaactgtttcagagtccacaaagagagaaaacatggacttttccatcagcatcagtgccatcaccccagcagatgccggcacctactactgtgtgaagttccggaaagggagccctgacacggagtttaagtctggagcaggcactgagctgtctgtgcgtgccaaaccctctgcccccgtggtatcgggccct

SEQ ID NO: 2 - protein, 125 aa, synthetic construct
EEELQVIQPDKSVSVAAGESAILHCTVTSLIPVGPIQWFRGAGPARELIYNQKEGHFPRVTTVSESTKRENMDFSISISAITPADAGTYYCVKFRKGSPDTEFKSGAGTELSVRAKPSAPVVSGP

SEQ ID NO: 3 - DNA, 45 bp, synthetic construct
  source 1..45: mol_type = other DNA
ggcggcggtgggagcggcggcggtgggagcggcggcgggggctcg

SEQ ID NO: 4 - protein, 15 aa, synthetic construct
GGGGSGGGGSGGGGS

SEQ ID NO: 5 - DNA, 1353 bp, synthetic construct
  source 1..1353: mol_type = other DNA
gaggtgcagctggtcgagagcggcgggggcctcgtgcagccgggcgggtcgctgcggctgagctgcgccgcgagcgggttcaacatcaaggacacctacatccactgggtgcgccaggcccccggcaagggcctcgagtgggtcgcccggatctaccccacgaacgggtacacccgctacgccgacagcgtgaagggccggttcaccatcagcgcggacacctcgaagaacacggcctacctgcagatgaacagcctgcgcgccgaggacaccgccgtgtactactgcagccggtggggcggcgacgggttctacgccatggactactgggggcagggcaccctcgtcaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacgccacgtaccgtgtggtcagcgtcctcaccgtcctgcaccaagactggctgaatggcaaggagtacaagtgcaaggtctccaacaaagccctcccagcccccatcgccgcaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaatga

SEQ ID NO: 6 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 645 bp, synthetic construct
  source 1..645: mol_type = other DNA
gacatccagatgacccagagcccgtcgagcctgagcgccagcgtgggcgaccgggtcacgatcacctgccgcgcgagccaggacgtgaacaccgccgtggcctggtaccagcagaagcccgggaaggcccccaagctcctgatctactcggcgagcttcctgtacagcggcgtccccagccggttcagcgggtcgcgcagcggcaccgacttcacgctcaccatcagcagcctgcagccggaggacttcgccacctactactgccagcagcactacaccacgccccccaccttcgggcagggcaccaaggtggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 8 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - DNA, 1773 bp, synthetic construct
  source 1..1773: mol_type = other DNA
gaggaggagctgcaggtgattcagcctgacaagtccgtatcagttgcagctggagagtcggccattctgcactgcactgtgacctccctgatccctgtggggcccatccagtggttcagaggagctggaccagcccgggaattaatctacaatcaaaaagaaggccacttcccccgggtaacaactgtttcagagtccacaaagagagaaaacatggacttttccatcagcatcagtgccatcaccccagcagatgccggcacctactactgtgtgaagttccggaaagggagccctgacacggagtttaagtctggagcaggcactgagctgtctgtgcgtgccaaaccctctgcccccgtggtatcgggccctggcggcggtgggagcggcggcggtgggagcggcggcgggggctcggaggtgcagctggtcgagagcggcgggggcctcgtgcagccgggcgggtcgctgcggctgagctgcgccgcgagcgggttcaacatcaaggacacctacatccactgggtgcgccaggcccccggcaagggcctcgagtgggtcgcccggatctaccccacgaacgggtacacccgctacgccgacagcgtgaagggccggttcaccatcagcgcggacacctcgaagaacacggcctacctgcagatgaacagcctgcgcgccgaggacaccgccgtgtactactgcagccggtggggcggcgacgggttctacgccatggactactgggggcagggcaccctcgtcaccgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacgccacgtaccgtgtggtcagcgtcctcaccgtcctgcaccaagactggctgaatggcaaggagtacaagtgcaaggtctccaacaaagccctcccagcccccatcgccgcaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaatga

SEQ ID NO: 10 - protein, 590 aa, synthetic construct
EEELQVIQPDKSVSVAAGESAILHCTVTSLIPVGPIQWFRGAGPARELIYNQKEGHFPRVTTVSESTKRENMDFSISISAITPADAGTYYCVKFRKGSPDTEFKSGAGTELSVRAKPSAPVVSGPGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 1065 bp, synthetic construct
  source 1..1065: mol_type = other DNA
gaggaggagctgcaggtgattcagcctgacaagtccgtatcagttgcagctggagagtcggccattctgcactgcactgtgacctccctgatccctgtggggcccatccagtggttcagaggagctggaccagcccgggaattaatctacaatcaaaaagaaggccacttcccccgggtaacaactgtttcagagtccacaaagagagaaaacatggacttttccatcagcatcagtgccatcaccccagcagatgccggcacctactactgtgtgaagttccggaaagggagccctgacacggagtttaagtctggagcaggcactgagctgtctgtgcgtgccaaaccctctgcccccgtggtatcgggccctggcggcggtgggagcggcggcggtgggagcggcggcgggggctcggacatccagatgacccagagcccgtcgagcctgagcgccagcgtgggcgaccgggtcacgatcacctgccgcgcgagccaggacgtgaacaccgccgtggcctggtaccagcagaagcccgggaaggcccccaagctcctgatctactcggcgagcttcctgtacagcggcgtccccagccggttcagcgggtcgcgcagcggcaccgacttcacgctcaccatcagcagcctgcagccggaggacttcgccacctactactgccagcagcactacaccacgccccccaccttcgggcagggcaccaaggtggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 12 - protein, 354 aa, synthetic construct
EEELQVIQPDKSVSVAAGESAILHCTVTSLIPVGPIQWFRGAGPARELIYNQKEGHFPRVTTVSESTKRENMDFSISISAITPADAGTYYCVKFRKGSPDTEFKSGAGTELSVRAKPSAPVVSGPGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1101 bp, synthetic construct
  source 1..1101: mol_type = other DNA
gaggaggagctgcaggtgattcagcctgacaagtccgtatcagttgcagctggagagtcggccattctgcactgcactgtgacctccctgatccctgtggggcccatccagtggttcagaggagctggaccagcccgggaattaatctacaatcaaaaagaaggccacttcccccgggtaacaactgtttcagagtccacaaagagagaaaacatggacttttccatcagcatcagtgccatcaccccagcagatgccggcacctactactgtgtgaagttccggaaagggagccctgacacggagtttaagtctggagcaggcactgagctgtctgtgcgtgccaaaccctctgcccccgtggtatcgggccctgcggcgagggccacacctcagcacgaattcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttga

SEQ ID NO: 14 - protein, 366 aa, synthetic construct
EEELQVIQPDKSVSVAAGESAILHCTVTSLIPVGPIQWFRGAGPARELIYNQKEGHFPRVTTVSESTKRENMDFSISISAITPADAGTYYCVKFRKGSPDTEFKSGAGTELSVRAKPSAPVVSGPAARATPQHEFEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - DNA, 1749 bp, synthetic construct
  source 1..1749: mol_type = other DNA
gaggaggagctgcaggtgattcagcctgacaagtccgtatcagttgcagctggagagtcggccattctgcactgcactgtgacctccctgatccctgtggggcccatccagtggttcagaggagctggaccagcccgggaattaatctacaatcaaaaagaaggccacttcccccgggtaacaactgtttcagagtccacaaagagagaaaacatggacttttccatcagcatcagtgccatcaccccagcagatgccggcacctactactgtgtgaagttccggaaagggagccctgacacggagtttaagtctggagcaggcactgagctgtctgtgcgtgccaaaccctctggcggcggtgggagcggcggcggtgggagcggcggcgggggctcgcaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctcaggacacagccatatattactgtgccagagccctcacctactatgattacgagtttgcttactggggccaagggactctggtcactgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtatgtggacggcgtggaggtgcataatgccaagacaaagccgcgggaggagcagtacaacgccacgtaccgtgtggtcagcgtcctcaccgtcctgcaccaagactggctgaatggcaaggagtacaagtgcaaggtctccaacaaagccctcccagcccccatcgccgcaaccatctccaaagccaaagggcagccccgagaaccacaggtgtacaccctgcccccatcccgggaggagatgaccaagaaccaagtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctccgggcaaatga

SEQ ID NO: 16 - protein, 582 aa, synthetic construct
EEELQVIQPDKSVSVAAGESAILHCTVTSLIPVGPIQWFRGAGPARELIYNQKEGHFPRVTTVSESTKRENMDFSISISAITPADAGTYYCVKFRKGSPDTEFKSGAGTELSVRAKPSGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 645 bp, synthetic construct
  source 1..645: mol_type = other DNA
gacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggatcccttccaggtttagtggcagtggatcagggacagattttactcttagcatcaacagtgtggagtctgaagatattgcagattattactgtcaacaaaataataactggccaaccacgttcggtgctgggaccaagctggagctgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 18 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 57 bp, synthetic construct
  source 1..57: mol_type = other DNA
atgggatggtcatgtatcatcctttttctggtagcaactgcaactggagtacattca

SEQ ID NO: 20 - protein, 8 aa, synthetic construct
GGGGGGGG

SEQ ID NO: 21 - protein, 15 aa, synthetic construct
GGGGSGGGGSGGGGS